(19) United States
(12) Patent Application Publication
(10) Pub. No.: US 2002/0052551 A1
(43) Pub. Date: May 2, 2002

(54) Systems and methods for tele-ophthalmology

(76) Inventors: Stephen H. Sinclair, Gladwyne, PA (US); Sanjay Bhasin, Ambler, PA (US)

Correspondence Address: PENNIE AND EDMONDS, 1155 AVENUE OF THE AMERICAS, NEW YORK, NY, 100362711

(21) Appl. No.: 09/935,424

(22) Filed: Aug. 23, 2001

Related U.S. Application Data
(60) Provisional application No. 60/227,192, filed on Aug. 23, 2000.

(51) Int. Cl. A61B 6/00

(52) U.S. Cl. 600/476; 128/920

(57) ABSTRACT

The present invention provides systems and methods for screening and tracking ophthalmic disease in a plurality of patients. The invention includes a screening subsystem comprising a non-mydriatic camera for obtaining digital images of eyes of the patients, a central database for storing the digital images of the eyes of patients as well as patient demographic data and related health data, and a central server comprising a computer which executes retinopathy grading algorithms, wherein the retinopathy grading algorithms recognize and assign a grade to ophthalmic disease present in the digital eye image and store the results in the central database. The invention also provides a method for screening and tracking ophthalmic disease in a patient with the steps of obtaining digital images of eyes of the patient by means of a screening subsystem comprising a camera, transmitting the obtained digital image to a central database and to a central server, executing retinopathy grading algorithms that recognize and assign a grade to ophthalmic disease present in the digital eye images, and storing the transmitted digital images and the results of the retinopathy grading algorithms in the central database.

The present application claims priority to and all benefits of U.S. provisional patent application serial No. 60/227,192 filed Aug. 23, 2000.

### 1. FIELD OF THE INVENTION

The present invention relates to systems and methods for providing ophthalmology services; in particular this invention relates to networked computer systems which provide for acquiring and screening retinal images for evidence of diabetic retinopathy and other ophthalmic diseases and for providing a database for tracking and analyzing ocular disease onset and progression.

### 2. BACKGROUND OF THE INVENTION

Retinal screening is an important ocular service. Many ocular diseases are progressive, and although their progress may be impeded or arrested, ocular damage once done cannot be reversed. Further, because slowly progressive vision impairment can be adapted to, vision problems may not be sufficiently perceptible to cause a patient to seek medical attention until the underlying disease is considerably advanced. Thus, early detection of ocular disease can be vitally important for preserving vision, and retinal screening of at least those at risk is key to early detection.

One devastating but prevalent and slowly progressive ocular disease is diabetic retinopathy (hereinafter “DR”), which is currently the leading cause of blindness in the United States and other developed countries. (American Diabetes Association PS., 1993, Clin. Diabetes 11:91-96.) Multiple studies indicate that most cases of severe vision loss as well as total blindness are due to a lack of adequate screening to detect retinal lesions early in the course of the disease. (Klein, 1997, Arch. Ophthalmol. 115:1073-1074) If not discovered until the patient has sufficient vision problems to initiate a visit to the physician, the disease most often is severely progressed and treatment, although successful at preventing further progression of the disease, is seldom able to restore lost vision. (Early Treatment Diabetic Retinopathy Study Research Group, 1987, Int. Ophthalmol. Clin. 27:265-272; Diabetic Retinopathy Study Research Group, 1981, Ophthalmology 88:583 et seq.) With the current diabetic population in the U.S. estimated at more than 14 million, and with more than 85-93% developing significant retinopathy within their lifetime, it is crucial to provide adequate and comprehensive screening for all diabetic patients on a regular basis. (Klein et al., 1987, Diabetes Care 10:633-638.)

Another ocular disease for which screening is advantageous is age-related macular degeneration (ARMD). Currently 38 million Americans age are over the age of 65; a number expected to increase to 80 million by 2020. The incidence of ARMD and blindness due to ARMD increases with age, from 2.7% of those over age 45, to 10% of those over age 65, to 20-30% or more of those over age 75. In fact, ARMD is the leading cause of blindness for those over the age of 65, and moreover leads to vision problems in over one-third of these individuals. Currently, ARMD is usually first discovered by physician examination, but only when it is too advanced for current treatments to restore vision, although in many cases further vision loss can be lessened. Therefore in order to adequately manage this blinding disease, regular screening is required with risk prediction to identify not only individuals at risk or eyes at risk, but also regions within one eye that may have a sufficient (threshold) risk. Currently, such management would require 20 million screening exams per year; a number expected to increase to more than 50 million exams per year by 2020.

Other conditions known to benefit from routine screening are glaucoma, detecting eye injuries such as laser injuries and their sequella, and so forth.

Known approaches for retinal screening include traditional eye examination performed by an ophthalmologist and evaluation by competent examiners of retinal photographs. Both these approaches require pupil dilation. These known approaches have at least three significant problems: variability of screening results, high cost of screening, and lack of patient compliance.

Traditional retinal screening during visual examination by an ophthalmologist is known to be expensive, because it requires highly trained medical personnel. (Kleinstein et al., 1987, J. Am. Optom. Assoc. 58:879-82.) The traditional method has also been found to lead to extremely variable results depending on the examining ophthalmologist. (Brechner et al. 1993, JAMA 270:1714-1718; Sussman et al., 1982, JAMA 247:3231-3234: Kraft et al., 1997, Arch. Fam. Med. 6:29-37.)

Screening of retinal photographs has similar problems. (Moss et al., 1985, Ophthalmology 92:62-67; Valez et al., 1987, Clin. Res. 35:363A.) Although nearly all of the pathology occurring, for example, in DR, may be captured in photographs of seven standard photographic fields, this screening method also is costly while leading to variable results due to examiner and photographic variability. (Only rare instances of pathology occur in the peripheral retina without accompanying posterior lesions.)

Attempts to improve photographic screening by creating “centralized reading centers” have in fact led to new problems while not ameliorating the previous problems. Centralized reading centers are central sites staffed by trained retinal graders to which remote sites send their film or digital photographs, obtained using non-mydriatic retinal cameras (i.e., that is cameras that do not require pupil dilation). First, delayed photograph interpretation prevents immediate quality control of photographs, while the patient is still at the remote site so that improved photographs may be taken if necessary. The patient may leave the remote site without a complete set of diagnostic quality screening photographs having been taken. Secondly, trained graders are also costly and have unacceptable variability, due largely to fatigue that reduces quality over the course of a day.

Another common problem with these present screening approaches is patient compliance. Retinal screening and early detection of retinopathy requires that a patient make yet one more appointment with another medical specialist for a condition that may not yet be a perceptible problem for the patient. Due to the protean nature of many diseases which affect the eye, e.g., micro and macro-vascular complications of DR, each such patient generally already has numerous appointments with numerous specialists. Investigations have demonstrated that compliance with established and known screening guidelines for early stage DR is no more than 35-50%, regardless of education and socio-economic level and type of health insurance coverage. (Brechner et al., 1993, JAMA 270:1714-1718; Donovan, 1995, Intl. J. of Tech. Assessment in Health Care 11:443-455; Sinclair et al., 1989, Invest. Ophthalmol. Vis. Sci. 30(S):79; Jacques et al., 1991, Diabetes Care 14:712-717.)

Thus, both traditional physician eye examination and human grading of retinal photographs, whether or not centralized, suffers from a lack of adequate quality control due to the significant variability among individual physicians and examiners. A direct consequence of this variability is that evaluation ocular disease progress over time is limited to an appreciation of only the most gross retinal changes. These approaches provide no useful mechanism in place for tracking disease progression. Further, both these approaches require use of trained and costly personal; and both approaches discourage patient compliance by requiring another medical appointment. In contrast, screening in a primary care office, which a patient may already frequent, or even at an unscheduled “walk-in” facility, improves compliance. Prior experiences with similar screening approaches have reported screening rates improved up to in excess of 83%. (Klein et al., 1997, Arch. Ophthalmol. 115:1073-1074)

Citation or identification of any reference in this Section or any section of this application shall not be construed that such reference is available as prior art to the present invention. Additionally, statements made in this section are not to be interpreted as admissions of prior art with respect to the present invention.

### 3. SUMMARY OF THE INVENTION

The present invention overcomes these problems in the prior art of vision case by providing simple, accessible, and economical screening methods and systems for a number of the most important retinal diseases, in particular for diabetic retinopathy (“DR”) and macular degeneration. Ocular screening systems (“OSS”) of the present invention include systems and methods designed to provide simple, convenient ocular screening for patients so that those with ocular disease are encouraged to have periodic screening. In this manner, vision loss can be slowed or halted. Although the number of routine retinal screenings is expected to be high, as much as possible of the screening process is automated, especially first level retinal image analysis, so that referral to expensive specialists need be made only for those with significant retinopathy. Thereby, this invention improves vision care while reducing its cost.

The present invention achieves these goals and objects by obtaining digital photographs of patients' eyes acquired with a non-mydriatic camera system (less preferably a mydriatic camera system) in a quality-controlled environment at conveniently located screening sites, and then by analyzing these images for retinopathy in an objective and quantitative manner at analysis center. The analysis center maintains a store of patient images for objectively tracking the retinal condition of individual patients, which also incidentally provides unparalleled resources for population studies of retinal diseases.

Because pupil dilation is not routinely required and because the automated image analysis is capable of rapidly screening and grading images, screening sites can offer complete examinations on a “walk-in” basis, requiring only 15-20 minutes for photography and retinal grading. Furthermore, the immediate identification at the screening site of those who have significant retinopathy coupled with a “closed loop” of physician communication between primary care physician, specialist, and ophthalmologist provided by the present invention contributes significantly to patient compliance with the follow-up investigation and treatment.

Significant elements of the systems and methods of the present invention include conveniently located screening sites and screening subsystems. Retinal cameras that do not require pupil dilation (non-mydriatic) provide sufficient quality images for OSS software evaluation. However, to ensure that the is sufficiently robust to allow retinal photography to be performed in a non-optometric/ophthalmologic setting by a non-ophthalmic technician, screening subsystems of the present invention are provided with a set of image quality assessment (“IQA”) algorithms that assures optimum quality by immediately evaluating each image upon acquisition for focus, contrast, pupillary alignment, and correct orientation. If the acquired images are of inadequate quality, the IQA algorithms provide immediate guidance to the technician for re-acquiring the images.

In this manner, the non-mydriatic (or mydriatic) screening system of this invention is capable of consistently producing reliable image quality for use in the automated retinopathy screening. Therefore, these screening subsystems objective may even be placed in the primary care setting in order to reduce the additional number of specialist appointments for the patient and to make the specialist appointments more appropriate to those who need the care.

Another significant element of this invention is one or more retinal grading algorithms that automatically evaluate the digital retinal images obtained by the screening subsystems for particular retinopathies. Generally, the RGAs operate in a lesion-based fashion, first identifying ophthalmologically significant retinal lesions or features by use of image processing methods, and second evaluating and grading the retinopathy in view of the identified lesions by use of artificial intelligence/cognitive decision capabilities. Because each retinopathy is usually characterized by a distinctive set of retinal lesions and features, each particular retinopathy advantageously has a separate set of RGAs with specialized image processing and decision capabilities. Preferably, the RGAs grade a patient's retinal images into least three grades comprising no retinopathy, or retinopathy that may be followed, or retinopathy that requires specialist examination.

RGAs are preferably executed on a high performance system shared by a number of screening sites (a central server) in order to rapidly prepare image evaluations at reduced cost.

Another significant element of the present invention is workflow management (“WFM”) facilities that, first, provide a comprehensive workflow environment that not only provides on-site screening with an assured level of image quality, but also provides for transmission of image data to central processing sites and to referral ophthalmologists where necessary. This transmission management function also provides for oversight that reports and evaluations are completed in a timely manner and are forwarded to those in need. Second, WFM facilities provide a “closed loop” scheduling environment of electronic messaging and reporting that facilitates communication between health care providers, offering a means to track the patient through screening, diagnosis, and treatment in order to insure patient compliance and to improve the outcomes for the patient's vision.

Importantly, the WFM facilities control image transmission, reporting, and messaging in dependence on a patient's ophthalmologic state determined by the system. For example, if RGA processing determines that images of a patient have third level retinopathy, the WFM facilities are informed and the images for this patient are transmitted for specialist review and evaluation. If the referral specialist so determines, further patient screening, examination, or treatment is managed by the WFM's closed loop scheduling environment.

Therefore, by means of the ophthalmologically responsive work flow management, it can be appreciated that specialist supervision of patients screened by systems of this invention is reserved for those truly in need. The greater majority with stable or less significant retinopathy are followed by periodic system re-screening until and if they require specialist examination.

Another significant element of the present invention is a centralized database (or a distributed database with a single image) (“CDB”) of all patient images, reports, demographic data, and other identifying information. This central database provides several surprising advantages to the systems and methods of the present invention. The longitudinal series of quantitatively analyzed retinal images of each patient, at least those who have been part of the system for some time, stored in the CDB permit for the first time (to the inventor's knowledge) the progress or regression of a patient's retinopathy to be viewed at the individual lesion level. Accordingly, this invention incorporates this objective historical lesion data into retinal grading, so that at least a retinal grade determined by a snapshot of the current retinal appearance may be revised based on the rate of progression or regression of the identified lesions. This leads to improved risk prediction for individual patients.

Further, image data in the CDB provide unparalleled information on retinopathies in the general population. Indeed, this information which for the first time is quantitative, in contrast to the qualitative impressions of treating ophthalmologist which have been all that was available until now. Population studies utilizing this data will provide, as elsewhere in medicine, improved quantitative understanding of retinal disease and lead to improved risk prediction factors and treatment outcomes.

Furthermore, the RGA algorithms have image processing and decision components both of which can advantageously be improved by use of training data, such as the images in the CDB. Therefore, this invention includes use of CDB image data to train and improve the retinopathy grading algorithms, and this use is expected to lead to sharp learning curve for the systems and methods of this invention.

The CDB also preferably stores administrative data, such as information identifying system screening sites and participating health care providers, and certain system data, such as rules controlling the WFM facilities.

Also significant is that the systems of this invention may be implemented in a cost effective manner in a client-server architecture. Points of physician access may be implemented by thin client which has web-browser and e-mail facilities. Screening sites need processing sufficient to acquire images and perform local image quality assessment. Most processing and data storage resources may be centrally implemented in a central server. In particular, the central server would make application processing available according to a known ASP model.

In summary, the present invention provides a non-mydriatic screening subsystems that are embedded in an overall IT infrastructure that is able to analyze the retinopathy in an objective and quantitative manner. It provides a reduced cost method of screening a larger population of patient in a more convenient scenario, ultimately improving patient compliance. It offers a measurable level of quality control by performing image quality analysis, as well as providing a “closed loop” environment within which all authorized medical personnel have access to image data and screening data/reports. With an improved means of patient monitoring through all phases of screening, diagnosis and treatment, it is believed that the overall goal of improved patient care is achievable. The consequences of not providing adequate screening for all patients having the potential for developing retinopathy has long-term societal costs in the follow-on care of severely vision-impaired persons.

In all embodiments, a mydriatic camera may be used in place of a non-mydriatic, especially in this instances where a mydriatic camera is already available.

In other words, the vast majority of patients do not require pupil dilation when retinopathy screening is performed with the OSS system. As a result, retinal screening compliance increases significantly when screening is provided in a closed-loop environment and available to the patient in the primary care setting as a ‘walk-in’ basis. Therefore, the OSS system provides a less costly method of performing retinal screening compared to the traditional methods of screening.

Finally, it should be reiterated that the present invention technology, although first focused on diabetic retinopathy, is applicable to a wide range of retinal and ocular diseases such as macular degeneration, glaucoma and laser induced-retinal injuries. Indeed, the technology is applicable even to diseases of organs other than the eye. Additionally, the present invention has applications where health and health care is provided large numbers of people, such as in industry, the military, and health management organizations.

In more detail, the present invention includes the following embodiments. In a first embodiment, the invention includes a method for acquiring one or more digital retinal images of adequate objective quality from a patient during a single image acquisition session, the method comprising: acquiring a digitally-encoded photographic image of a retinal field in an eye of the patient with a retinal camera, determining one or more objective quality measures for the acquired digitally-encoded image by processing the image with one or more image quality assessment algorithms, wherein the image is determined to be of adequate quality if all the objective quality measures are determined to be adequate, repeating the steps of obtaining and determining only if one or more of the determined quality measures are determined to be inadequate, wherein, prior to repeating the step of obtaining, instructions are provided to adjust the retinal camera in a fashion to correct inadequate quality measures, and wherein the repetitions, if any, of the steps of obtaining and determining are limited by the duration of the image acquisition session.

In aspects of the first embodiment, the invention further includes that the step of repeating is limited to at most three repetitions of the steps of obtaining and determining; that the one or more objective quality measures determined by the image quality assessment algorithms are correct image orientation, or correct level of image contrast, or correct image focus, or absence of image edge flare; that (i) if image orientation is inadequate, then the provided instructions comprise visual mis-alignment examples and corrective actions relating to the relative rotation of the camera and the eye, (ii) if image contrast is inadequate, then the provided instructions comprise corrective actions relating to the relative anterior-posterior position of the camera and the eye, (iii) if image focus is inadequate, then the provided instructions comprise corrective re-focusing actions, and (iv) if absence of image edge flare is inadequate, then the provided instructions comprise corrective actions relating to the relative X-Y position of the camera and the eye.

In a second embodiment, the invention includes a system for acquiring one or more digital retinal images of adequate objective quality from a patient during a single image acquisition session, the system comprising: a retinal camera, a computer including a processor and memory which is coupled to the camera for image transfer to the memory, and wherein the memory is provided with instructions encoding the steps of receiving into the memory from the camera a digitally-encoded photographic image of a retinal field in an eye of the patient, processing the image with one or more image quality assessment algorithms which determine one or more objective quality measures for the image, wherein the image is determined to be of adequate quality if all the objective quality measures are determined to be adequate, and repeating the steps of obtaining and determining only if one or more of the determined quality measures are determined to be inadequate, such that (i) wherein, prior to repeating the step of obtaining, instructions are provided to adjust the retinal camera in a fashion to correct inadequate quality measures, and (ii) wherein the repetitions, if any, of the steps of obtaining and determining are limited by the duration of the image acquisition session.

In aspects of the second embodiment, the system further includes that the one or more objective quality measures determined by processing the image with quality assessment algorithms are correct image orientation, or correct level of image contrast, or correct image focus, or absence of image edge flare

In a third embodiment, the invention includes a computer program product for acquiring one or more digital retinal images of adequate objective quality from a patient during a single image acquisition session, the product comprising at least one computer-readable memory with encoded instructions for receiving into a memory of a computer from a camera a digitally-encoded photographic image of a retinal field in an eye of the patient, processing the image with one or more image quality assessment algorithms which determine one or more objective quality measures for the image, wherein the image is determined to be of adequate quality if all the objective quality measures are determined to be adequate, and repeating the steps of obtaining and determining only if one or more of the determined quality measures are determined to be inadequate, such that (i) wherein, prior to repeating the step of obtaining, instructions are provided to adjust the retinal camera in a fashion to correct inadequate quality measures, and (ii) wherein the repetitions, if any, of the steps of obtaining and determining are limited by the duration of the image acquisition session.

In a fourth embodiment, the invention includes an automatic method for grading one or more digitally-encoded images of a retinal field of an eye of a patient with respect to a selected retinopathy, the method comprising: processing the digitally-encoded retinal image to detect, identify, and characterize in the retinal image lesions from a pre-determined set lesion types, wherein the pre-determined set of lesion types describe visual features characteristically found in retinas with the selected retinopathy, performing a decision process that assigns a grade to the retinal image in dependence of on properties of the detected lesions.

In aspects of the fourth embodiment, the system further includes that the retinal image includes information at two or more wavelengths, and that the step of processing detects, identifies, and characterizes lesions in the retinal image with wavelength-dependent properties in dependence on the wavelength information; that the retinopathy is diabetic retinopathy, that the pre-determined lesion types include micro-aneurysms, or dot hemorrhages, or blot hemorrhages, or striate hemorrhages, or nerve fiber layer infarcts, or lipid exudates, or cotton wool spots, or neovascularization; that the decision process assigns (i) a first grade if no lesions are detected, (ii) a second grade if only one or more micro-aneurysms are detected, (iii) a third grade if one or more micro-aneurysms and one or more of dot hemorrhages or of blot hemorrhages or of striate hemorrhages are detected, and (iv) a fourth grade if one or more micro-aneurysms and one or more of dot hemorrhages or of blot hemorrhages or of striate hemorrhages and one or more of nerve fiber layer infarcts or of lipid exudates or of cotton wool spots or of neovascularization; that the step of processing further comprises: detecting potential lesions as identified image features not discriminated as normal retinal features, detecting probable lesions as detected potential lesions with geometric configurations and pixel variability thresholds fitting a type of pre-determined lesion, detecting lesions by a decision process based on image features, geometric configurations, pixel variability thresholds, and signature features of the detected probable lesions, wherein the signature features include texture parameters and spectral characteristics.

In aspects of the fourth embodiment, the system further includes that the step of performing, the properties of the detected lesions comprise their identities, their numbers, their sizes, and their retinal positions; that the retinal positions comprise positions with respect to the optic nerve head and the fovea; that the steps of processing and performing include one or more decision processes, and wherein the method further comprises a step of training the decision processes including: assigning grades to the plurality retinal images from patients having the selected retinopathy by performing a manual grading method, assigning grades to a plurality retinal images from patients having the selected retinopathy by performing the automatic method of this embodiment, and adjusting the decision processes so that the grades assigned by the automatic method are of adequate accuracy in comparison to the grades assigned by the manual method.

In a fifth embodiment, the invention includes a system for grading one or more digitally-encoded images of a retinal field of an eye of a patient with respect to a selected retinopathy, the system comprising: a computer including a processor and memory wherein the memory is provided with a digitally-encoded retinal image, and wherein the memory is further provided with instructions encoding the steps of detecting, identifying, and characterizing lesions in the digitally-encoded retinal image from a pre-determined set of lesion types, wherein the pre-determined set of lesion types describe visual features characteristically found in retinas with the selected retinopathy, and executing a decision process that assigns a grade to the retinal image in dependence of on properties of the detected lesions.

In aspects of the fifth embodiment, the system further includes that the instructions encoding the steps of detecting, identifying, and characterizing further encode the steps of detecting potential lesions as identified image features not discriminated as normal retinal features, detecting probable lesions as detected potential lesions with geometric configurations and pixel variability thresholds fitting a type of a pre-determined lesion, detecting lesions by a decision process based on image features, geometric configurations, pixel variability thresholds, and signature features of the detected probable lesions, wherein the signature features include texture parameters and spectral characteristics.

In a sixth embodiment, the invention includes a computer program product for grading one or more digitally-encoded images of a retinal field of an eye of a patient with respect to a selected retinopathy, the product comprising at least one computer-readable memory with encoded instructions for detecting, identifying, and characterizing lesions in a digitally-encoded retinal image from a pre-determined set of lesion types, wherein the pre-determined set of lesion types describe visual features characteristically found in retinas with the selected retinopathy, and executing a decision process that assigns a grade to the retinal image in dependence of on properties of the detected lesions.

In a seventh embodiment, the invention includes a method for grading one or more digitally-encoded images of a retinal field of an eye of a patient taken at a selected time with respect to a selected retinopathy, the method comprising: processing the digitally-encoded retinal image taken at the selected time to detect, identify, and characterize in the retinal image lesions from a pre-determined set lesions type, wherein the pre-determined set of lesion types describe visual features characteristically found in retinas with the selected retinopathy, processing at least one digitally-encoded retinal image of the patient taken at least one time prior to the selected time to detect, identify, and characterize in the prior retinal images lesions from the pre-determined set lesions type, comparing the lesions detected in the image taken at the selected time with the lesions detected in the prior image to detect changes in the lesions, and performing a decision process that assigns a grade to the retinal image taken at the selected time in dependence on the identities and characteristics of the lesions detected in that image, and in dependence on the changes in the lesions detected in the comparing step.

In an eighth embodiment, the invention includes a system for grading one or more digitally-encoded images of a retinal field of an eye of a patient taken at a selected time with respect to a selected retinopathy, the system comprising: a database including at least one digitally-encoded retinal image of the patient taken at at least one time prior to the selected time, a computer including a processor and memory which is coupled to the database and wherein the memory is provided with a digitally-encoded retinal image, and wherein the memory is further provided with instructions encoding the steps of detecting, identifying, and characterizing lesions in the digitally-encoded retinal image taken at the selected time from a pre-determined set of lesion types, wherein the pre-determined set of lesion types describe visual features characteristically found in retinas with the selected retinopathy, retrieving into memory the digitally-encoded retinal image of the patient taken at the prior time, detecting, identifying, and characterizing lesions in the retrieved retinal image taken at the prior time from the pre-determined set lesions type, comparing the lesions detected in the image taken at the selected time with the lesions detected in a prior image to detect changes in the lesions, and performing a decision process that assigns a grade to the retinal image taken at the selected time in dependence on the identities and characteristics of the lesions detected in that image, and in dependence on the changes in the lesions detected in the comparing step.

In a ninth embodiment, the invention includes a 20. An automatic method for annotating one or more digitally-encoded images of a retinal field of an eye of a patient with respect to a selected retinopathy, the method comprising: processing a digitally-encoded retinal image to detect, identify, and characterize in the retinal image lesions from a pre-determined set lesion types, wherein the pre-determined set of lesion types describe visual features characteristically found in retinas with the selected retinopathy, annotating the retinal image with indicia indicating at least the positions of the detected lesions.

In aspects of the ninth embodiment, the system further includes that the annotation further indicates characteristics of the detected lesions; steps of retrieving the retinal image to be processed from a database of retinal images prior to the step of processing, and storing the annotated retinal image in the database subsequent to the step of annotation; that prior to the step of retrieving: receiving the retinal image to be processed from a source of retinal images, and storing the retinal image to be processed in the database.

In a tenth embodiment, the invention includes a method for managing the retinal screening of a patient likely to have a retinopathy comprising: receiving at least one digitally-encoded retinal image taken from the patient, receiving a grade for the retinal image from automatic retinal grading methods scheduled to evaluate the received retinal image, performing a decision process according to which if the grade indicates the presence of significant retinopathy, then receiving a further grade for the retinal image from manual grading methods scheduled to evaluate of the retinal image, or if the grade indicates the presence of retinopathy but not significant retinopathy, then scheduling to receive at least one retinal image taken from the patient after a selected first interval, or if the grade indicates the presence of retinopathy but not significant retinopathy, then scheduling to receive at least one retinal image taken from the patient after a selected second interval.

In aspects of the tenth embodiment, the invention includes that the step of receiving further comprises acquiring the retinal image from a retinal camera, and evaluating by image quality assessment algorithms whether the image's quality is adequate for the automatic retinal grading methods; that, if the received image is indicated to have an inadequate quality for the automatic retinal grading methods, then further performing a step of receiving a grade for the retinal image from manual grading methods scheduled to evaluate of the retinal image; that the first interval is selected in dependence on the severity of the retinopathy indicated by the grade, and wherein the second interval is selected to be longer than the first interval

In aspects of the tenth embodiment, the invention includes the step of transmitting a reminder message if a grade has not been received from scheduled manual grading methods with a selected time period; the steps of receiving a referral message from a health care professional requesting screening for the patient, scheduling receipt of a retinal image taken from the patient, and transmitting a reminder message if an image has not been received with a selected time period.

In an eleventh embodiment, the invention includes a system for managing the retinal screening of a patient likely to have a retinopathy comprising: a database, a computer including a processor and a memory which is coupled to the database and enabled to receive digitally-encoded retinal images, wherein the memory is further provided with instructions encoding the steps of (i) receiving into the memory at least one digitally-encoded retinal image taken from the patient, (ii) scheduling automatic retinal grading methods scheduled to evaluate the received retinal image, the automatic retinal grading methods returning a grade for the retinal image, (iii) performing a decision process according to which if the grade indicates the presence of significant retinopathy, then receiving a further grade for the retinal image from manual grading methods scheduled to evaluate of the retinal image, or if the grade indicates the presence of retinopathy but not significant retinopathy, then scheduling receipt at least one retinal image taken from the patient after a selected first interval, or if the grade indicates the presence of retinopathy but not significant retinopathy, then scheduling receipt at least one retinal image taken from the patient after a selected second interval, and (iv) storing in the database the received retinal image, information returned from the automatic retinal grading methods, and information generated by the performed decision process.

In aspects of the eleventh embodiment, the invention includes that the received retinal image is taken at a selected time, wherein the database stores at least one digitally-encoded retinal image of the patient taken at at least one time prior to the selected time, and wherein the instructions encoding the automatic retinal grading methods encode the steps of detecting, identifying, and characterizing lesions in the digitally-encoded retinal image taken at the selected time from a pre-determined set of lesion types, wherein the pre-determined set of lesion types describe visual features characteristically found in retinas with the selected retinopathy, retrieving into memory the digitally-encoded retinal image of the patient taken at the prior time, detecting, identifying, and characterizing lesions in the retrieved retinal image taken at the prior time from the pre-determined set lesions type, comparing the lesions detected in the image taken at the selected time with the lesions detected in the prior image to detect changes in the lesions, and performing a decision process that assigns a grade to the retinal image taken at the selected time in dependence on the identities and characteristics of the lesions detected in that image, and in dependence on the changes in the lesions detected in the comparing step.

In aspects of the eleventh embodiment, the invention includes one or more systems according to claim 5, wherein the system according to claim 5 are enabled to transmit the retinal images to the computer; one or more access means for health care professionals, wherein the access means provide for receipt of reports and for transmission of requests concerning the patient by health care professionals

In a twelfth embodiment, the invention includes a computer database comprising one or more computer readable media with a database constructed according to the method of the ninth embodiment. Also, in all embodiments, a mydriatic camera or a non-mydriatic camera may be used to obtain retinal images.

### 4. BRIEF DESCRIPTION OF THE FIGURES

The present invention may be understood more fully by reference to the following detailed description of the preferred embodiment of the present invention, illustrative examples of specific embodiments of the invention and the appended figures in which:

FIGS. 1A-B illustrate general embodiments of the systems and methods of the present invention (wherein “primary care physician/specialist/diabetologist” is abbreviated as “PCP/SPC/DBT”);

FIGS. 2A-B illustrate general embodiments of a screening center of the present invention;

FIGS. 3A-E illustrate general embodiments of the central server processing of the present invention; and

FIG. 4 illustrates general embodiments of the physician access of the present invention.

### 5. DETAILED DESCRIPTION OF THE PREFERRED EMBODIMENT

Preferred general embodiments of the systems and methods of the ophthalmology service system (referred to herein as the “OSS”) of the present invention are first described; followed in subsequent sections by descriptions of the principle preferred components of the general embodiments.

5.1. Systems and Methods

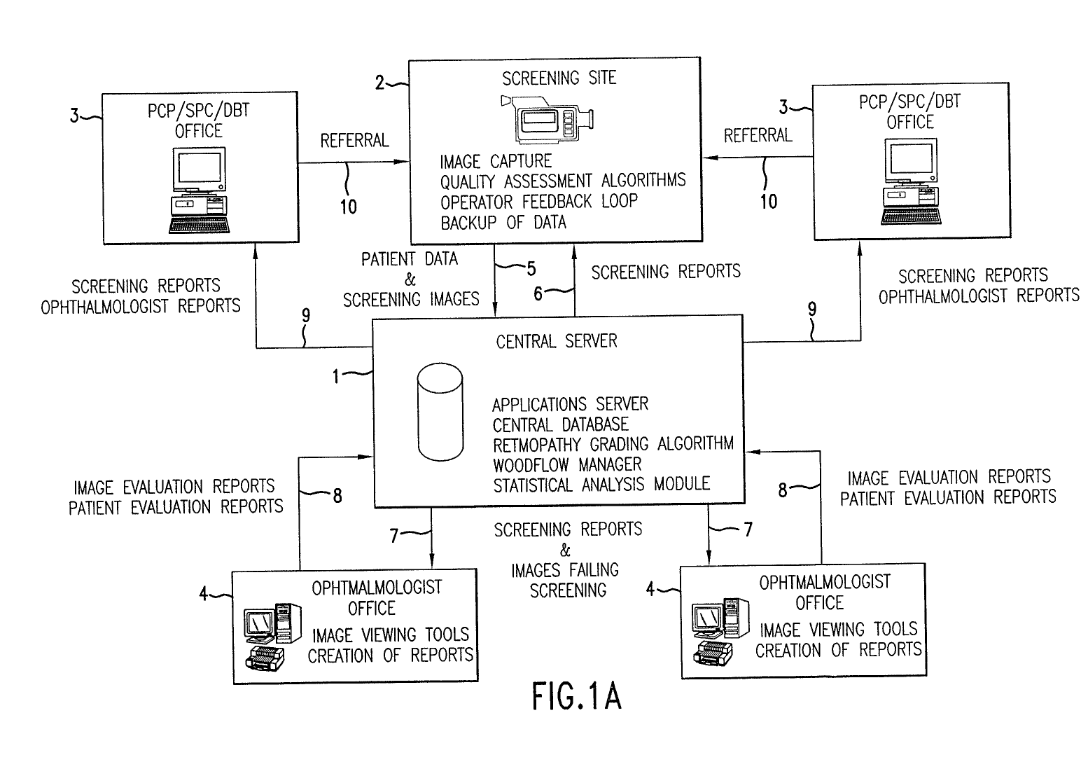

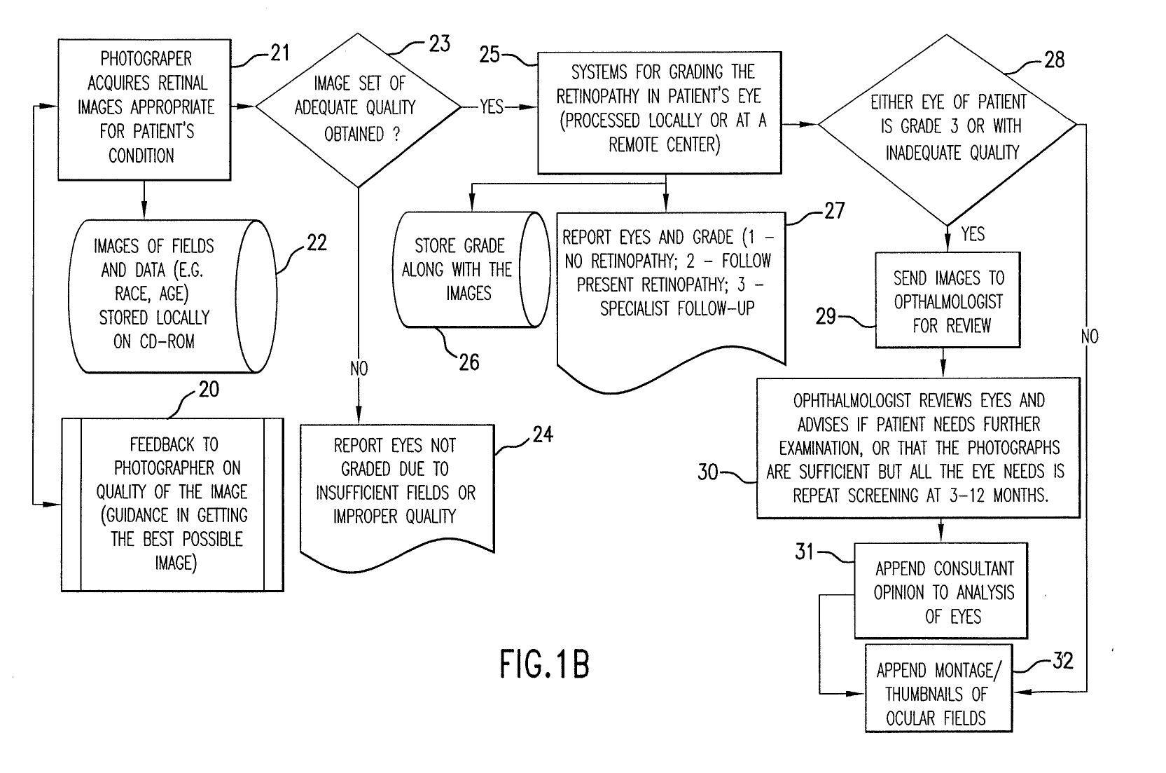

As illustrated in FIG. 1A, the overall OSS architecture includes central server 1, which provides application services (e.g., by an ASP model) including retinopathy grading algorithms, statistical and patient analysis, and workflow management, and which houses a central database containing patient demographic information, all patient image data, screening results, and reports. This server repository is fed information from a network of geographically distributed screening sites 2, which capture ocular images guided by local image quality algorithms and operator feedback and also backup locally patient data. The screening sites are preferably located in primary care settings that are frequented by patients, such as, in the case of diabetics, diabetic clinics. Although the intent is that the retinal screening be done at the same site as the point of care, the architecture is such that all components act independently and may be dispersed. In particular, data can originate from diverse sources, including optical shops.

Also part of the OSS architecture, and preferably present in an OSS system, is online physician access. A patient's direct care providers preferably access the system by means of access facilities in their offices 3, for example, PC-type systems networked to the system components, or by means of various portable or handheld communication devices. Direct care providers may include primary care providers as well as specialists who manage aspects of a patient's condition that may have ocular side-effects. A common example of the latter specialists, for diabetics, are diabetologists, because virtually all diabetics eventually develop diabetic retinopathy to some degree, and nephrologists, for similar reasons and because progression of retinopathy is known to be associated with progression of nephropathy. Also, a patient's ophthalmologist, who does not otherwise participate an OSS system, may access it in this fashion.

However, ophthalmologists who participate in an OSS system have office access facilities 4 that preferably have high bandwidth access to the central server, and provide high resolution image viewing tools and report creation facilities. Batch image transmission may serve in place of high bandwidth access. These ophthalmologists screen and evaluate ocular images that failed automated screening in the central server, or images in which automated screening detected serious abnormalities. Optionally, for periodic system test and quality assurance, or even generally, these ophthalmologists may review all images to insure the accuracy of automated screening.

The flows of image data and medical requests and reports among these components of an OSS system are indicated by the arrows in FIG. 1A, and are explained with reference also to FIG. 1B, illustrating the overall methods of the present invention. Conventionally, patients enter an OSS system by referral from their direct care provider, whether generalist or specialist. This referral requires nothing more than presenting a paper prescription or paper referral form to a screening site or making a telephone call. However, preferably, referral takes the form of electronic messages exchanged with components of an OSS system (either a screening site as illustrated in FIG. 1A, or the central server, not illustrated), so that the system itself may determine the most convenient screening site for the patient, which may be in another health care facility, or in an optical shop) or even next door in the direct care provider's offices, or so forth.

Once a patient arrives for screening at a screening center, after identification, demographic, “clip-board” style medical history (paper or electronic) are obtained and entered into the system (if not already done), the image acquisition processes commence. The actual images acquired are dependent on the ocular disease present, because different diseases present in different anatomic regions and layers of the eye. For example, because diabetic retinopathy (hereafter “DR”) rarely presents with peripheral retinal lesions in the absence of fundus (central retinal) lesions, the inventors have discovered that images of no more than five selected fields within the fundus are adequate to screen DR.

Importantly, image are acquired to the greatest extent possible using non-mydriatic cameras (i.e., cameras not requiring pupil dilation) are used because mydriatic is inconvenient for the patient, requiring a recovery time, and mydriatic cameras (i.e., cameras requiring pupil dilation) are expensive. The invention is immediately applicable to mydriatic cameras, however. Further, obtaining a gradable set of ocular images on the first screening-center visit prevent the inconvenience of return visits. To achieve these objects, image acquisition 21 (FIG. 1B) is coupled with immediate automatic assessment of image quality providing feedback to the photographer that the acquired image is of adequate quality or that the image needs to be reacquired. In the latter case, the quality assessment algorithms provide indications of the image quality problems along with suggestions for correction.

If a set of correct images of adequate quality are obtained 23 (FIG. 1B), perhaps after a few reacquisitions, they are transmitted 5 (FIG. 1A) along with patient data for grading at central server 1. If, after a permitted number of reacquisitions, images of adequate quality cannot be obtained, a report 24 of this result is also transmitted 5 to central server 1. Optionally in cases of failure with non-mydriatic camera, if a screening center has a mydriatic camera available and if the referring physician so permits on the referral, image acquisition may be attempted after mydriasis. This is preferred for those conditions, e.g., cataracts, abnormally small pupil, that may benefit from mydriasis.

After image acquisition and transmission, next central server 1 executes grading algorithm (FIG. 1B) appropriate to the patient's ocular condition and creates screening report 27. Preferably, communication and computation resources are adequate so that the automatic grading may be executed and the screening report may be transmitted 6 (FIG. 1A) back to the screening site before the patient leaves the site. It is believed that such immediate feedback will motivate the patient to carry out the actions recommended in the report. Further, the central server stores all information, e.g., original images, screened and interpreted images, patient data, and any reports, in the central database (also the “CDB”).

Preferably, the retinopathy grading algorithms (hereinafter “RGA”) for a particular condition provide at least a three level grading. According to this preferred grading, an image is graded as: level 1, no retinopathy recognized for the condition; level 2, retinopathy recognized but screening in a shortened interval recommended; and level 3, significant retinopathy recognized with specialist consultation recommended. At a minimum the RGAs grade into two levels, namely a first level where periodic screening is recommended and a second level where specialist consultation recommended. The preferred grading (or the minimum grading) permits an OSS to achieve its object of providing patient screening at the recommended intervals while referring only those patients in need for specialist examination.

More preferably, the retinopathy grading algorithms (hereinafter “RGA”) have sufficient algorithmic robustness to provide a retinal grade for an retinal image approximating clinical grading currently used for that ocular condition being examined, or alternately to provide a grade which reflects the extent of the recognizable lesions. In such an embodiment, an OSS may make more refined patient recommendations reflecting the increased grading resolution. For example, in contrast to the “condition independent” recommendations provided in the three level grading embodiment, a more preferable may provide more refined recommendation as a function of the grading and the specific ocular condition. Recommendations may be made by an expert system, which may be rule-based, that reflects ophthalmic practice for the condition when a particular retinal grade is determined. It is also preferred that the RGAs produce a screened image or the equivalent in which recognized lesions characteristic of the condition are marked on the image. Additionally, or alternately, a list of lesions identifying, i.e., their type, their position on the retina, their size, and so forth, can be appended to the retinal images in the CDB.

Also, in preferred embodiments where prior retinal images are available and may be compared to a current retinal image, the time progression or regression of lesions may be identified. Then, detailed lesion information and lesion history may be taken into account in adjusting retinal image grading. For example, if a current image received a grade of level 2, but it contained lesions in critical anatomic regions as near the optic nerve head, or the fovea, or so forth, or contained rapidly growing or multiplying lesions, it may be promoted to grade level 3. Conversely, if a current image received a grade of level 3, but it contained regressing lesions in locations posing no threat of imminent visual impairment, it may be demoted to grade level 2 (or 2+) (Generally, grade level 3 signifies specialist consultation is recommended, while grade level 2 signifies that routine follow-up screening is recommended.)

Next, if a patient has a level 3 image from either eye 28 (FIG. 1B), the entire set of images are transmitted 7 (FIG. 1A and 29 in FIG. 1B) to participating ophthalmologist 4. The participating ophthalmologist reviews the images confirm a level 3 grade or perhaps to change the grading to level 2 with more or less frequent follow-up screening. The report of the examining ophthalmologist is then transmitted 8 back to the central server. This human review step is preferred and prudent in cases of potentially serious retinopathy. Also, it is prudent and preferable for a participating ophthalmologist review images with inadequate quality for automatic screening to assess these patients also for the presence of serious retinopathy.

Finally, the central server assembles a final patient report including. preferably, the automatic screening report, the ophthalmologist report (if any) 31, and a montage or thumbnails of the recent images 32. The final report is then transmitted 9 to the office 3 of the direct care physician.

Additionally, the workflow manager component of the central server notifies the patient and the direct care physician of recommendations and arranges to the extent possible repeat screening in the case the images of adequate quality were not obtained, follow-up screening at the appropriate interval in the case of a level 2 grade, and specialist appointment in the case of a level 3 grade.

Although the invention is described herein in a preferred embodiment as methods and systems including elements for performing ophthalmic screening and folloe-up for a plurality of patients, the present invention also includes useful “sub” embodiments including one or only a few of the elements present in the complete system. For example, the screening subsystem or its methods alone are useful embodiments to obtain retinal images; the retinal grading algorithm methods and systems performing these methods alone are useful embodiments to grade retinal images; the workflow manager methods and systems performing these methods alone are useful embodiments to manage ophthalmic patients; and so forth. Moreover, useful combinations and sub-combinations of elements of the present invention apparent to those of skill in the art are included within its scope even though not explicitly described herein.

Additionally, where useful, all embodiments include program products including encoded instructions to carry out the methods or implement the systems as well as computer readable media including data used and created by these embodiments. The computer readable media can be any such media known in the art, such as, magnetic disks and tapes, optical disks, even download over a network.

The present invention is described in more detail in the following with respect to the individual architecture system and method components introduced above. Although preferred embodiments are described, variations of the preferred embodiments that will be immediately apparent to those of skill in the are intended to be within the scope of the present invention. For example, although the OSS is described with its functions distributed among a number of dispersed components, other distribution of function are possible. In one alternative distribution, a small OSS may include a single merged screening site and central server for serving only a single or only a few physician offices.

5.2. Screening Site

Screening sites have one or more screening subsystems, which include the camera, hardware, and software for the input into an OSS of patient identification and demographic data (for new patients) as well as of acquired retinal images. A single screening subsystem should be capable of screening preferably 8, 12, or 16 patients per day, and a single screening site may sufficient screening subsystems to handle patient volume. Each screening subsystem performs the following general functions:

Entry of a patient into the OSS system;

Image Capture of retinal images;

Image Quality Assessment algorithms;

Operator feedback loop;

Transmission of images to Central Database;

Backup Process;

Ability to print hard copy of images.

For image capture and acquisition, preferably a non-mydriatic retinal camera is used to acquire retinal images in order to avoid the patient inconvenience of pupil dilation (mydriasis). It has been discovered that for most conditions five images (three to seven), each of about a 45° field (25° to 45°) and acquired for each eye screened, have adequate quality for analysis by the RGAs, even when the non-mydriatic camera system is operated by a non-ophthalmic-trained technician. The RGAs are advantageously specifically adapted for the images acquired by non-mydriatic cameras, preferably in view of a sufficiently large database of retinal images of at least approximately retinal images from 4,000 eyes. Further, non-mydriatic cameras have the additional advantage of being less costly than commercially available mydriatic cameras.

The present invention may use a wide range of non-mydriatic cameras, including commercially-available cameras from, e.g., Canon, Nikon, and so forth, and also including specially designed and built cameras. From whatever source, preferred cameras have should have optics capable of acquiring up to 45° retinal fields through pupils down to 2.0 mm in diameter with adequate image contrast and resolution. Images should be captured at least a 3K×3K×32 3-color bit resolution, for example, by commercially available three chip CCD sensors such as are available from Sony, and so forth. The CCD sensor electronics should provide high speed image transfer to associated computer hardware using such standard interfaces as USB, IEEE 1832, Firewire, or so forth.

Controls for camera focus and orientation should permit easy, convenient, and intuitive camera manipulation even by non-professional (but trained) operators. Controls preferably include infrared monitoring of focus and orientation and an internal fixation array or fellow-eye fixation array to assist with proper eye positioning for each field. Physical positioning of the camera controls is important, for example advantageously a joystick can control camera elevation, lateral movement, and exposure. A control for switching from the iris viewing lens to the retinal viewing lens may be positioned near the joystick.

However, it may be advantageous for at least some screening sites to also have a mydriatic camera for those patients whose ocular conditions require, and whose referring physicians have prescribed, pupil dilation, or from whom image of adequate quality cannot be obtained for whatever reason.

An image handling system associated with (one or more) a camera may simply include a standard PC-type computer, for example a Pentium based PC running a Windows operating system (NT or 2000). The system should have a quality monitor so that the operator may view clearly important anatomic landmarks in the retina and the image to be acquired. Each screening site also preferably has high-bandwidth (i.e., DSL or similar) links to the central server, or at least a 56K or faster modem. Also preferable are a color inkjet printer and a writing device for CD-ROMs or DVD-ROMs.

Screening Site Methods

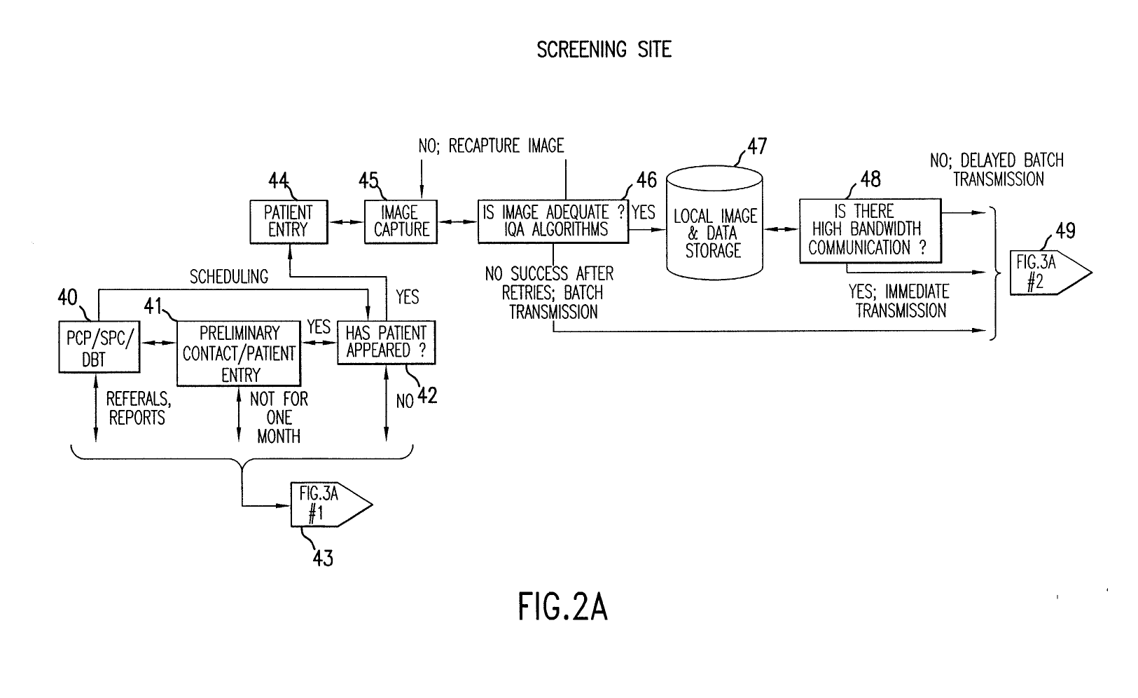

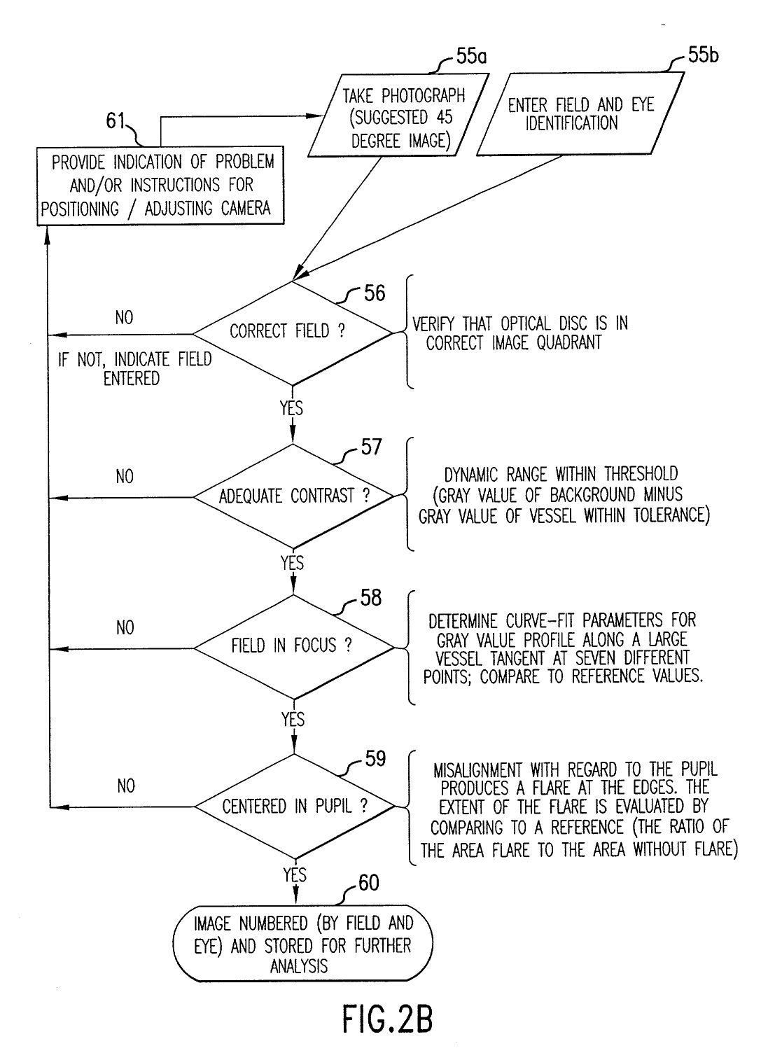

In one embodiment, a screening site in cooperation with the central server performs methods such as those illustrated in FIG. 2A. OSS processing for a patient begins when the patient's direct care physician (a primary care physician, a specialist, a diabetologist, or so forth as the case may be) refers 40 (FIG. 2A) the patient to the system for screening. In one embodiment, the referral may be accomplished by a ‘prescription/referral’ form and encourages the patient to have screening done immediately. In another embodiment, the referral may be accomplished by exchange of electronic messages (or by telephone) from the physician office access with the OSS. Preferably, the system then schedules the patient for the most convenient screening site. Once the patient appears at a screening site, patient identification, demographic, and physician information is entered 41 into the system preferably by means of simple graphical user interfaces. The system also includes a check for appearance 42 of a patient at or within a certain window of the scheduled screening appointment. If the patient has not appeared, the system generates reminder for the patient's physician and preferably also for patient.

These patient management steps cooperate (indicated by off-page connector 43) with the central (or global) workflow manager (hereinafter, the “WFM”) so that patient status is maintained system-wide, and not screening-site-by-screening-site. Thus, the global WFM is aware of patient schedules, patient information, patient appearances at any site in the system, and so forth, so that there is thorough follow-up of patient appointments and screening results. The patient may be screened at any screening site or seen at any physician's office (depending on the scope of the network connected, even world wide) without loss or duplication of management and information.

After patient appearance and entry 44, image capture 45 commences. In an exemplary embodiment, a complete set of retinal images includes non-stereo, 45° images (alternatively, 30°, 40°, 45°, 50°, or 60° images depending upon retinal camera) of five fields of each eye, a total of ten images. Preferred but exemplary fields include the following:

| | |
|---|---|
| field #1OD: | disc visible at right margin of field (fovea at center), |
| field #2OD: | disc visible at lower right margin (supero-temporal), |
| field #3OD: | disc visible at lower left margin (supero-nasal), |
| field #4OD: | disc visible at upper left margin (infero-nasal), |
| field #5OD: | disc visible at upper right margin (infero-temporal), |
| field #6OS: | disc visible at left margin of field (fovea at center), |
| field #7OS: | disc visible at lower right margin (supero -nasal), |
| field #8OS: | disc visible at lower left margin (supero-temporal), |
| field #9OS: | disc visible at upper left margin (infero-temporal), and |
| field #10OS: | disc visible at upper right margin (infero-nasal) |

(where “OD” designates the right eye, and “OS” the left eye). (Alternatively, a field centered on macula, on the optic disc at top, on the optic disc at far right, on the optic disc at bottom, and on the optic disc at far left may be used.)

Shortly after each image is captured, its quality is assessed 46. If the image quality is inadequate, the operator is instructed to re-capture the image; otherwise, if the quality is adequate, the operator moves on to the next image. Most importantly, real-time determination of image quality ensures that the patient does not leave the screening site without a complete set of adequate-quality images. This avoids the considerable inconvenience to the patient or possibly multiple repeat visits until adequate-quality images are obtained, as well reducing screening costs. Further, the immediate feedback to the operator is an exceptional training tool that improve the image capture technique of the operator and thus the quality of captured images.

When a complete set (preferably ten) of adequate-quality images are acquired, they are sent to the central database (“CDB”) (indicated by off-page connector 49) along with the patient demographic data, and results of the image quality assessment analysis by the highest bandwidth link available 48, for example, DSL. This transmission is preferably in real-time, but may by ‘batch’ process during off-hours over slower links. Images of inadequate quality are transmitted at a lower priority for any analysis that may be possible. Each screening site also stores 47, at least for back up, patient data and images. Back-up local storage is preferably low cost, such as writeable CD-ROM storage.

For certain patients with significant degrees of lens or media opacity, image of adequate quality may not be achievable with non-mydriatic cameras. For such patients (estimated at approximately 10-12%), pupil dilation may be preferably (if permitted or prescribed), and they are preferably photographed with mydriatic cameras at screening sites with a back-up mydriatic camera available. Advantageously. a subset of the images of those patients who required dilation may be examined by a retinal specialist to determine if any adjustments to the image quality assessment algorithms are necessary. For example, the scoring mechanism might be too stringent or too relaxed, or modifications to the operator training may be required in the case of excessive dilation. In some fewer cases, image acquisition may not succeed even with dilation or pupillary dilation may be inadequate even with mydriatics.

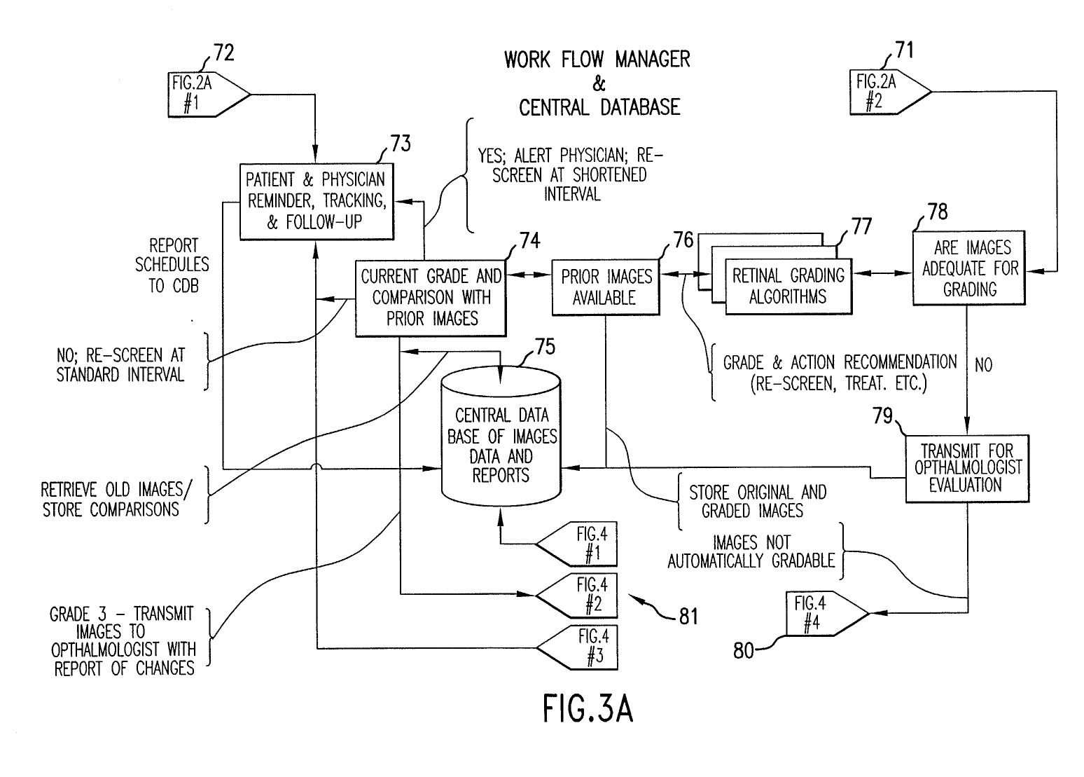

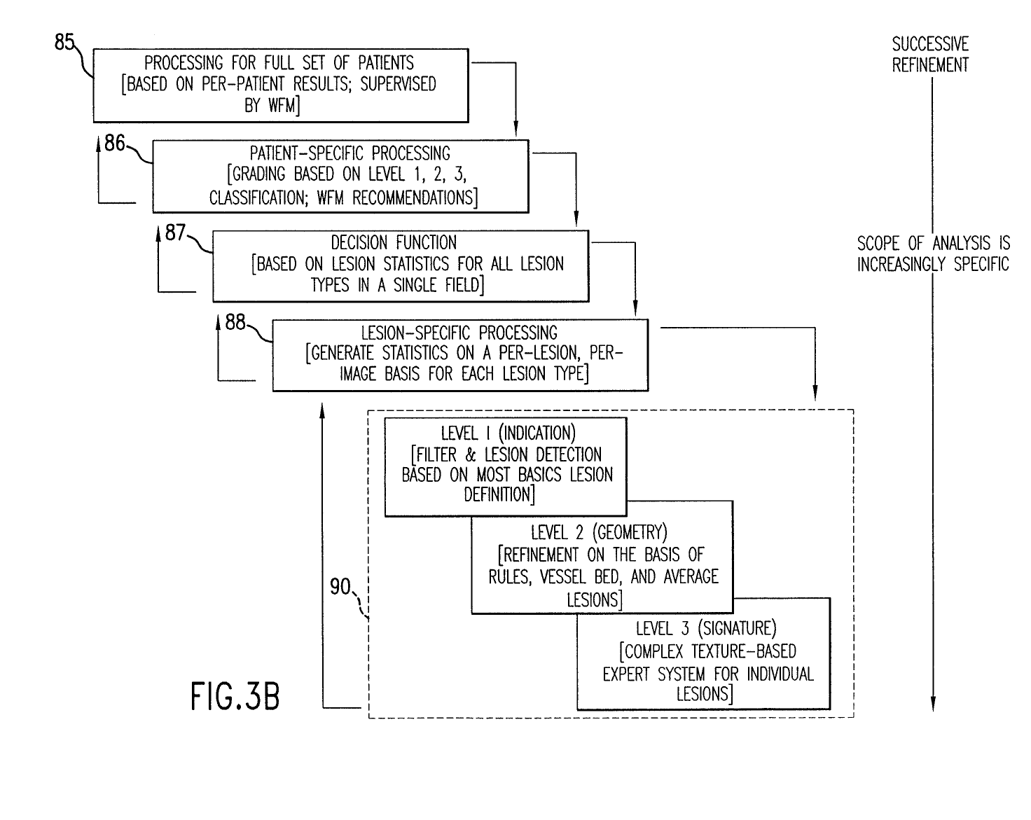

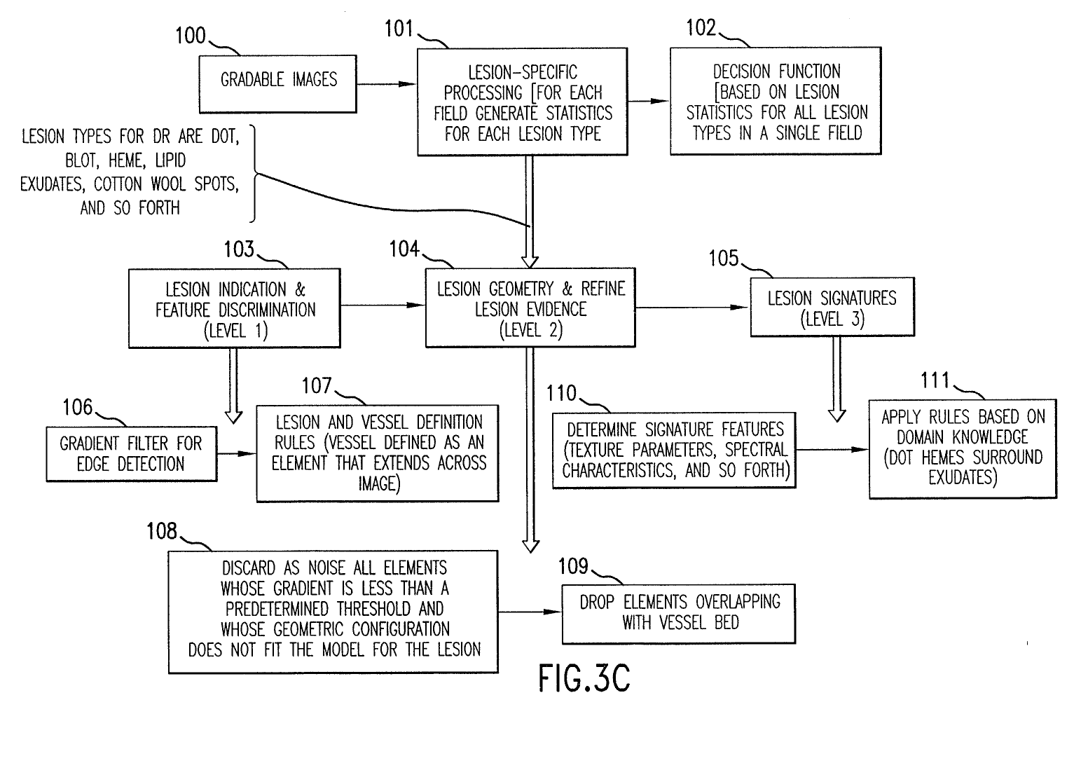

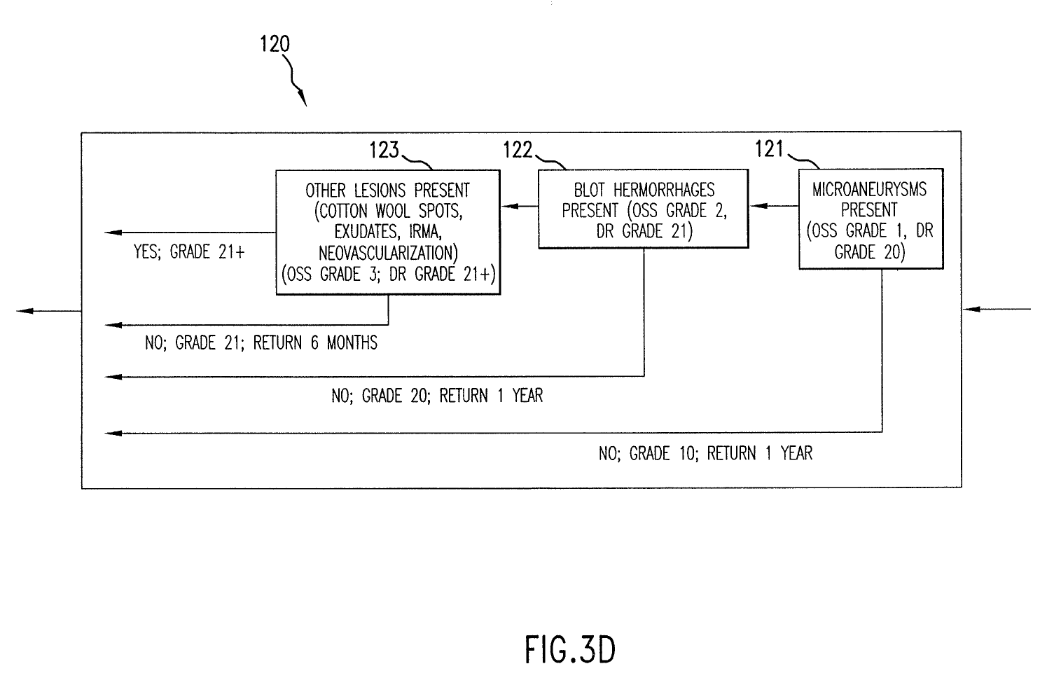

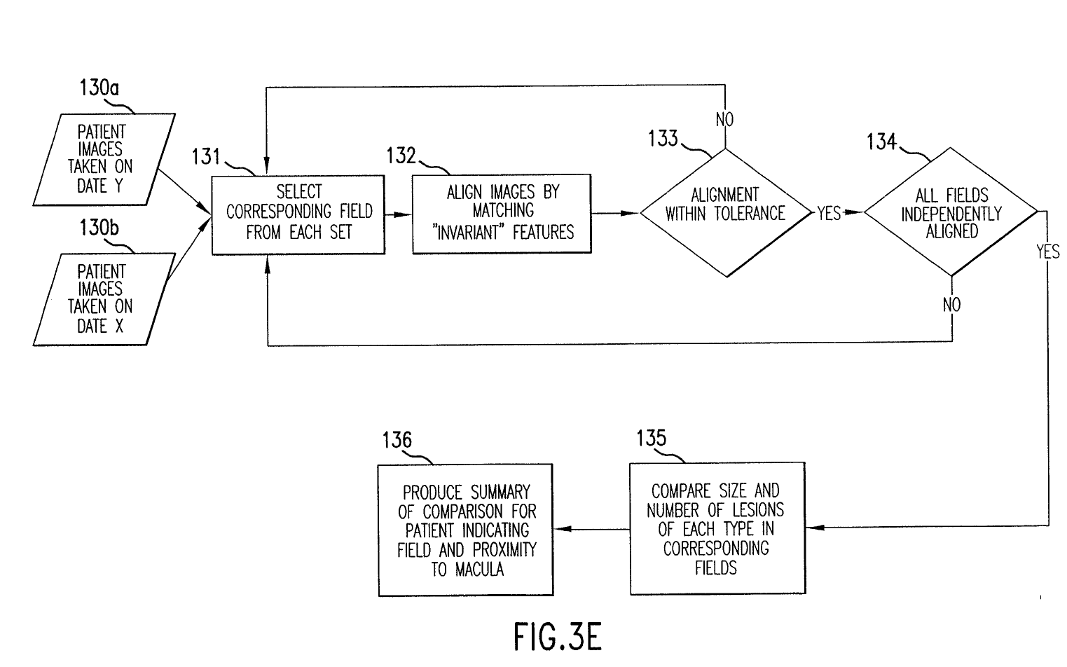

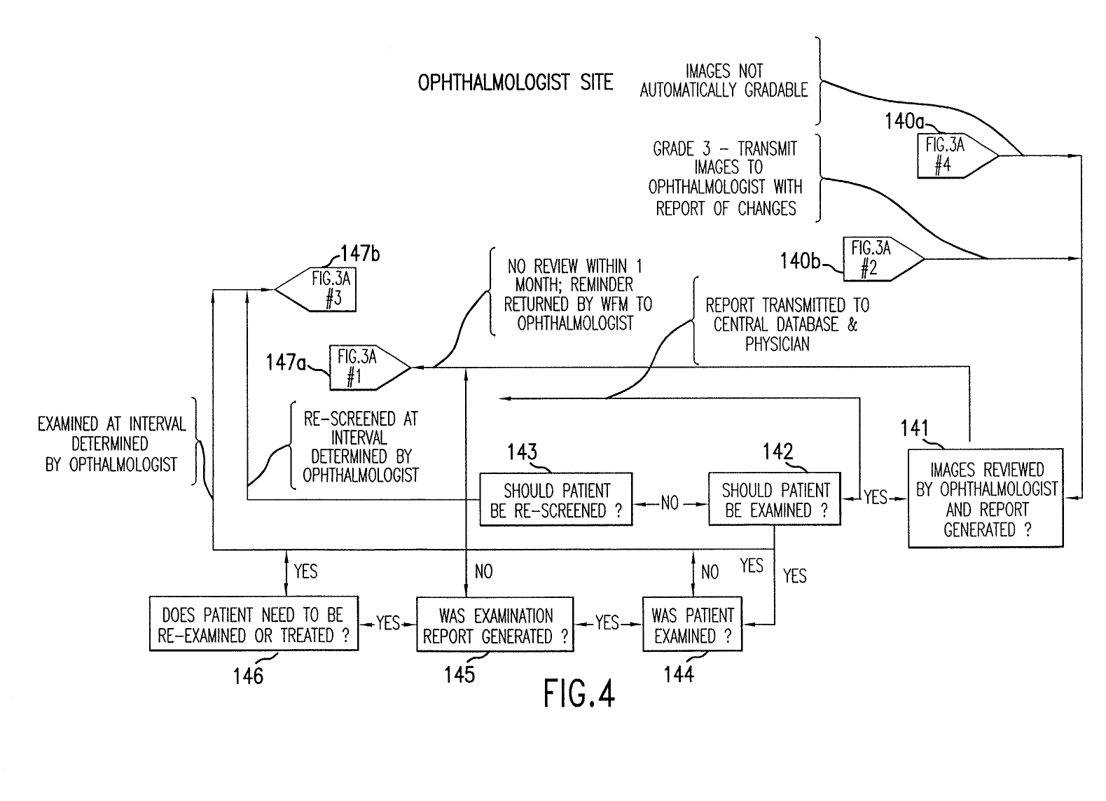

The images of these patients, after transmission to the central server, are automatically transmitted by the workflow manager on to a participating ophthalmologist (along with images of eyes with more advanced retinopathy) for manual evaluation (step XXX in FIG. 3A). Further, when image quality is inadequate, this workflow manager saves pertinent information relative to the cause of failure (cataracts, pupil less than 3.5 mm in the infrared focusing light, pupil size after pharmacological mydriasis, etc.).

5.2.1. Image Quality Algorithms

Importantly, and significantly promoting patient convenience and reducing cost, this invention includes image quality assessment (hereinafter “IQA”) algorithms that run locally on the screening subsystem (or on a server at screening site) enabling each image to be and analyze the quality of each image shortly, or even immediately, after acquisition. If the image does not meet the required quality, the screening subsystem interacts with the operator to give guidance as to the reason for failure and possible ways to improve the quality by, for example, camera adjustment and positioning. For example, if a flare-type artifact has been identified, the operator is recommended to shift the camera slightly to one side (with respect to the pupil) as dictated by the position of the flare. This interactive and interative process is continued until adequate images are acquired, or until a maximum number of retries (for example, for three retries). Optionally, if all images are insufficient in quality for any of the fields, the operator is instructed to dilate the eye (if permitted) and retake the images.

Preferably, the IQA algorithms function by analyzing each image against certain rapidly determinable criteria. For DR, a preferably set of criteria include correct photographic orientation, level of contrast, existence of image edge flare, and image focus. For other ocular conditions additional criteria may be advantageous, for example, level on contrast in two or more wavelength bands. In further embodiments, the set of criteria may be selected by the IQA algorithms for each image according to the patient's diagnosed condition.

Preferred IQA algorithm methods are illustrated in FIG. 2B. Although preferred, FIG. 2B is exemplary at least in that in other embodiments the order of testing the criteria may be different, and further certain criteria may be added or the illustrated criteria removed. Further, the criteria tested may be patient and/or condition dependent. The IQA algorithm begins after the operator takes a photograph 55a of an ocular field and enters the field and eye identification 55b of the image. The first test 56 made is whether the correct field has be photographed. For each field in each photographed eye, the eye must be rotated and oriented such that the proper portion of the retina is photographed. Fundus orientation is easily checked by examining the intended view and then orienting the camera so that the actual view being returned from the camera matches key features of the intended view. For example, a easily identified feature that may be used for matching is the optic nerve head. A set of visual mis-alignment cases along with the corrective measure will be provided to the photographer to guide the process.

The next test 57 is of image contrast. Contrast may be reduced because incorrect anterior-posterior position of the camera leads to a too dark or too light image. For example, the image may be too dark when the camera is positioned too far from the eye, or may be too light when the camera is positioned too close. A too-light image may also result from light reflected from the iris, for example consequent to inadequate pupil dilation. The IQA algorithms evaluate a criteria that operationally reflects image contrast, namely the ratio of retinal vessel (vein) contrast to background contrast at green wavelengths. Vessel contrast is measured from the fall-off of a brightness histogram of pixel values along a line that is perpendicular to the edge of a vessel. Background contrast brightness is measured by widths of brightness histograms of, for example, three sample regions away from the fovea after application of a median filter. A median filter of sufficient size will reduce contrast and remove contributions to the histogram from objects.

Image focus is tested next by identifying retinal vessels and then examining the cross section of a number of vessel(s) for blur at the margins in the green wavelengths (maximum absorption for oxygenated and deoxygenated blood). Vessel cross sections are found in an image using the vessel identification by thresholding, gradient operators, and line following algorithms for the purpose of constructing a signature for image matching. (It is known in the image processing arts that gradient operators and equivalents are sensitive to spatial variations in pixel intensity.) Gross measures in a region will also be used based on Markov Random Fields for self-characterization into 4-5 distinct bands or classes, the resulting clusters being examined for extent and width across the spectrum of values.

The last test 59 evaluates image flare (seen as a peripheral arc of increased luminosity and reduced contrast) that may be produced at the edge of the photograph when the camera is improperly positioned with respect to the pupil (X-Y positioning). Flare is caused by light reflection in an arc from the iris, and is most severe when the eye is torted in order to photograph an eccentric portion of the retina causing the pupil to become ellipsoid with a narrowed axis in the direction of eye torsion. The peripheral flare in the picture is in the direction of misalignment over the iris (i.e., if the peripheral arc of flare is in the upper left position, the camera has been positioned too far up and to the left and must be moved down and to the right to take a subsequent photograph without the edge flare).

Preferably, flare is detected by searching for artifacts in the corners of an image. An extraction algorithm detects and outlines the portion of the image belonging to the eye, and the eccentricity of the extracted portion is measured. For example, diameters of the extracted portion are measured in several directions along lines are drawn through the portion's geometric center, and are compared to determine the extracted portion is approximately circular. Any deviations from circularity indicate flare in the image. If the flare is appreciable the image is rejected; if not appreciable, regions of flare are blocked from the image, and it is accepted. Alternatively, the algorithms examine the background of the picture for uniformity of the luminosity and contrast, and if flare is detected, the photographer is instructed to move the camera in the direction opposite to the flare. In another alternative, two-level segmentation using K-means to clustering finds the portion of the image without flare, and the ratio of this portion to total image size is determined along with the ratio of perimeter to area of the portion.

If an image passes tests 56-59, then is stored for grading 60. If any test is failed, the results of the tests are provided to the operator. Preferably, also, the results are interpreted 61 by an expert system, for example, a rule-based system or a neural network, that determines directions for the operator to retake the photograph. Based on the camera, specific directions will be provided to adjust the camera positioning and orientation to obtain a better photograph.

Basic image processing is known art described in many textbooks, such as, e.g., Russ, 1999 3^{rd} ed., The Image Processing Handbook, CRC Press LLC, Boca Raton, Fla.

5.3. Central Server

An OSS system according to the present invention presents patient and physician users with consistent and unified system-wide workflow management (i.e., patient scheduling, automatic report and data distribution, and so forth), patient data storage, and patient image storage. In a preferred embodiment, such a consistent image is achieved by a localized central server system, illustrated by server 1 in FIG. 1A. However, those of skill in the art will appreciate other implementations capable of presenting a consistent image that are also within the scope of this invention. For example, central-server functions may be performed by a distributed system implementing a distributed database and distributed workflow management. Such a distributed system may advantageously include several, linked server nodes, each individual node specialized to provide central-server functions for a selected geographical region. With such specialization, it is anticipated that inter-node traffic and the overhead of maintaining a distributed single-system image is minimized. because patient tend to seek medical case within their own home regions the great majority of the time. However, for concreteness of description, the central server has been described, and is described herein, in the preferred localized cental-site embodiment.

As illustrated in FIG. 1A, the central server hosts at least the following basic application components: the Central Database (“CDB”), Retinopathy Grading Algorithm (“RGA”), Workflow Manager (“WFM”) and Statistical Analysis Module (“SAM”). These applications are structured according to an Applications Service Provider (“ASP”) model, which allows all health care providers participating in an OSS network to access patient data and image through a simple web-enabled application to be run on their existing personal computers.

The central server ASP model preferably supports direct HTTPS requests, as well as HTTP requests where security is not required, from a user (such as a physician) via a standard web browser interface. Login and password entry are required. Users invoke the applications provided by the central server by requesting dynamic web pages or forms, and providing input through standard XML or HTML forms. Applets, such as JAVA servlets, executed on the Central Server accept input requests from the user's browser (e.g., a request for a patient's report or images) and respond by providing the contents for the browser (e.g., delivering the patient's report or images). As well as serving static and dynamic web-pages, the multithreaded server of the ASP manages the database access, security, and transaction services such as listening to the network for client requests, and establishing connections with a client, including negotiating details such as protocol, encryption and authentication.

The model allows resulting user accessibility using ‘thin-client’ hardware to all types of data (image & reports) with little administrative overhead required at the remote sites.

The central server may be implemented with conventional hardware and software. For example, hardware may be, or equivalent to, a Dell PowerEdge 4400, Pentium® III XeonT processor, 733 MHz or faster, 1GB RAM, 54GB Ultra SCSI Hard Disk with RAID and tape backup. For increased performance, multiprocessor servers or networked servers may be used. Software includes server operating systems such as Microsoft Windows NT 4.0 or 2000 server or a unix such as Linux. Database software is preferably a commercial database management system supporting SQL92, such as the Oracle8i or the equivalent. Applications may be coded in any convenient language, such as C++, Java, and so forth.

5.3.1. Central Database

The central database (“CDB”) is an on-line (or otherwise efficiently accessible) storage repository of the data generated in an OSS system. The CDB stores patient oriented data such as original image data from patient screening examinations, results of RGA screening including images annotated or marked-up with lesion identification, associated patient identification, demographics, and screening/examination history, results of manual ophthalmologist grading process including any annotated images, referrals and reports. This database also stores system oriented data such as statistical data gathered from analysis of the patient data, results of the image quality assessment process, the ‘rules’ to be used by the WFM for handling images, reports, and messages.

Image data requires the great majority of CDB storage, and the amount of image data to be stored may be estimated from the number of screening examinations to be stored. Currently, a standard screening examination acquiring ten images generates approximately 10-15 MB of data. This amount is likely to increase with increase in camera resolution and so forth. If compression is to be applied to stored image data, it must be rigorously verified to be lossless; accurate review of stored images may be required at any time. CDB storage facilities are advantageously scalable to accommodate growth over time.

The CDB has several uses in an OSS, and its centralized image (also possible with distributed database architectures) provides several advantages. Its principal use is to provide physicians, specialists, ophthalmologists, and other users with access to current images as well as the results of any prior studies, regardless of where acquired. This historical record permits an objective and quantitative evaluation, either by automatic algorithmic processes or by manual physician examination, of the status and progression of the ocular disease in individual patients. To the inventors knowledge, this is the first systematic method data by which such historical data has been applied to management of ocular disease.

The CDB may also be used to develop new analysis methods for ocular disease. For example, the images stored in this database are an invaluable resource for developing and testing new lesion detection and grading algorithms. For example, for grading vascular diseases of the eye, such as DR, algorithms measuring vascular tortuosity, branching angle, caliber variation, and so forth are important although hitherto unavailable. Such algorithms can enhance risk prediction, predominantly in the early stages of DR. Such detailed parameters are not accessible to human grading because of its qualitative nature. Further, use of historical image series in the CDB permit development of objective risk prediction algorithms.

Also, at a population level, data mining of the CDB allows screening proficiency and patient compliance to be examined, provides valuable insight into the trends within various populations, and allows treatments to be objectively assessed.

Next, for concreteness, an exemplary and non-limiting catalog of certain major CDB divisions, and of the types of data in each division, is presented.

Patient Division

(1) Permanent patient data

Identification (name, address, telephone number, e-mail address, SS number, billing information, date of birth, database identification number, and so forth)

Diagnoses (ICD9 code, duration, severity)

Physician information (treating primary care physician, specialist physician, ophthalmologist)

(2) Patient data entered following each screening session

Screening session identification (screening site identification, date, time, confirmation of patient data, race (affects image processing parameters), sex, photographer, camera utilized and type of images acquired)

Acquired Images (all fields from both eyes, image quality assessment)

Image grading results and reports (automatic grading of both eyes, all fields, grade levels; manual grading results if image unsuitable for automatic grading or if significant retinopathy is present)

Grading results include any graded, annotated, or marked-up images

Lesion data (type, severity, size, location)

System recommendations generated

Referral ophthalmologist's report if any (ophthalmologist recommendations)

Screening Site Division

Identification (address, hours of operation, operators present, and so forth)

Equipment available (cameras, other resources)

Local screening site database—(each screening site maintains certain local data)

Each site has mirror of its division data

Local storage of images acquired at site

Certain data for patients screened mirrored from the CDB

Physician Division

Identification (name, address, speciality)

5.3.2. Workflow Manager

The workflow manager (“WFM”) is for many purposes the processing hub of a system according to the present invention. It is responsible for processing referrals and scheduling patients, for routing data, reports, messages, and images among the various users of the system, for triggering other processing such as executing the appropriate RGAs for newly received screening images, for tracking expected user responses and actions and issuing reminders if expected actions are delayed.

FIG. 3B illustrates an embodiment of the overall processing method of an OSS and the WFM's role in this processing. In one aspect, at highest level 85, OSS processing involves checking for work to be done. Thus, the WFM may periodically scan and review the full set of system patients and physicians, evaluates their status against its processing rules, and schedules events and activities as necessary. For example, if a patient scheduled for a screening examination has not appeared at a screening site within a specified period, the WFM schedules reminders to be sent to the referring physician, and perhaps to the patient also. Further, if a participating ophthalmologist, who has been referred images for evaluation, has not returned a screening report in an agreed upon period, the WFM schedules a reminder that the report is overdue. This periodic scanning generally involves performing patient/physician specific processing 86 on many or all patients/physicians.

In another aspect of OSS processing, the WFM may also trigger patient/physician specific processing 86 for specific patients/physicians when a new event enters the OSS systems. For example, upon acquisition of a new set of adequate-quality images for a patient, the WFM triggers at least RGA image processing 90 (described in more detail subsequently). RGA processing preferably returns at least a system image grade and optionally a disease-specific, clinical grade. Preferably, RGA processing also returns marked-up and evaluated images and lesion-specific information which is processed 88 as directed by the WFM. Furthermore, when an ophthalmologist returns a report for a set or images not automatically gradable, similar information is extracted and similarly processed.

A central and important feature of WFM processing, whether initiated by periodic scan or by event arrival, is decision function processing 87. Here, the WFM takes and schedules actions based on ophthalmologic criteria and data. For example, recommendations are made for further patient and physician action based on a grade determined for recent screening images. If screening images reveal stable or clinically-low-grade retinopathy, then recommend further periodic screening. On the other hand, if the screening images reveal advancing or clinically-high-grade retinopathy, warn at least the physician and schedule specialist referral and examination.

In a preferred embodiment, these WFM decisions are represented by rules, each rule indicating one or more processing, communication, or scheduling actions for the WFM to take when a specified condition or event (or combination of conditions and events) is observed. Rule may be stored in a database of rules, for example, in a division of the CDB. The following are exemplary rules.

Grade evaluation: if (current system grade 3 & lesions regressing over time), then (lower current system grade to 2+)

Grade evaluation: if (current system grade 2 & lesions progressing over time), then (raise current system grade to 2+ (or to 3 if rapid progression))

Recommendation: if (current system grade 3), then (recommend specialist examination/consultation)

Recommendation: if (current system grade 2) then (recommend re-screening at shorter interval)

Recommendation: if (current system grade 1), then (recommend re-screening at longer interval)

Clinical adjustment: case (current disease), select (make disease-specific adjustments to grade thresholds, intervals, and other WFM parameters)

Communication: if (patient no-show & previous system grade 3), then (send warning message to physician/patient)

Communication: if (patient no-show & previous system grade 2), then (send alerting message to physician/patient)

Communication: if (report not returned from ophthalmologist in agreed interval), then (send reminder message)

One of skill in the art will realize that these listed rules are merely exemplary and non-limiting. The WFM, and an OSS system generally, may, of course, utilize many further rules of greater specificity and more varied functions.

Importantly, as is apparent, in order to manger activities in an OSS system, the WFM of this invention necessarily responds to ophthalmologic information from various sources.

In another aspect, the hierarchical WFM processing described implements a tracking mechanism for a community of health care providers responsible for the care of patients with primary or secondary ocular disease, providing, i.e., a “closed loop” system of patient care. FIG. 3A illustrates this aspect in more detail.

Patient information collected at the time of screening, such as the referring physician and ophthalmologist, communications with the direct care physicians, such as referrals and screening reports, and communications with participating ophthalmologists, such as evaluation of poor quality images and image with more severe retinopathy, are received by the WFM (indicated by off-page connector 72 to the physicians' offices and screening sites, and by connectors 81 to participating ophthalmologists offices). These reports and information are processed 73 by the WFM, preferably according to stored rules, as described. Processing results are stored in CDB 75, and further schedules, recommendations, and messages may be generated and returned to these offices and sites. Preferably message and reports are exchanged sent electronically; however the system may use conventional fax or mail per preference.

When images are received from a screening site (indicated by off-page connector 71), the WFM first determines 78 whether or not they are automatically gradable. If they are not, the WFM refers and transmits 79 them for manual grading by participating specialist/ophthalmologist (indicated by off-page connector 80), who returns reports and evaluated images (indicated by off-page connectors 81). If they are of adequate quality, the WFM invokes RGA processing 77, selecting the particular algorithm appropriate to the patient's ocular diagnosis. After RGA processing of the current images, the WFM checks whether or not prior images 76 for this patient are available in the CDB. If so they are retrieved, and the WFM combines current and historical information in a (rule-based) ophthalmologic decision process 74. WFM decisions are finally stored in the CDB 75 and typically communicated to OSS users 73.

Stated differently, in a clinical situation, the appropriate RGA evaluates the images and determines the level of retinopathy. If significant retinopathy is detected, the image data and screening results are then routed to the patient's ophthalmologist. Alternately, the WFM may notify the patient's ophthalmologist of any screening results, but may automatically route image data only if system retinopathy grade 3 (or DR grade 21+) was detected. The ophthalmologist then promptly reviews the images and determine whether the patient should be seen in the near future, or should be screened on a more frequent interval with photography.

To report the retinopathy grade level for each eye (or “non-gradable”), a structured reporting form (optionally, XML-based) can be advantageously used via a web interface, i.e., a ‘check-off’ template indicating findings. The report is automatically transmitted to and stored in the CDB and also routed to the patient's primary care physician as well as for backup. If the patient has system grade 3 (DR grade 21+) retinopathy, ‘ungradable’ photographs in either eye, or if a specialist (for diabetes, the diabetologist) requests, the screening report and images is forwarded to the designated ophthalmologist. In such cases, a reporting form is also included (electronic or paper depending upon the mode of transmission) with a request for the ophthalmologist to indicate the outcome of his review. The ophthalmologist is advised to return the reporting form to the central server (which is forwarded to the specialist), and to print a copy of his report to be filed as part of the patient's chart. It is believed that this method minimizes the ophthalmologist time involved compared to the traditional method of reporting, and speeds the dissemination of information throughout the patient care network.

If the ophthalmologist does not send the report to the CDB within a specified time interval from receipt of the image data, a reminder is generated by the WFM and sent to the ophthalmologist. If a follow-up screening was recommended by the ophthalmologist, and the patient has not returned to any screening site in the network within the recommended time interval, the WFM initiates a reminder be sent directly to the patient (printed and sent through the mail or sent electronically by e-mail) as well as to the specialist.

Thus, the WFM has the ability to route the information to the appropriate destination at the proper time. The environment of the present invention provides a means of enhanced collaboration for patient monitoring between primary care physicians/diabetologists, ophthalmologists/retinal specialists, and other specialists such as nephrologists who are responsible for treatment of co-disease aggravating factors. The overall benefit of the ‘closed loop’ system is increased patient compliance because of increased convenience and decreased cost and therefore improved patient care.

OSS/WFM for Diabetic Retinopathy

Herein is described a specific OSS implementation for diabetic retinopathy (“DR”) caused by diabetes mellitus (“DM”). This implementation is exemplary and non-limiting, and it intended only as an applied example of the work flow methods of this invention. This implementation includes primary care or direct care physicians (“PCP”), a screening site, retinopathy grading algorithms (“RGA”) which are optionally executed in a central server, and participating and non participating ophthalmologists. Each of these elements and their data flow is now described.

Primary Care Physician

The PCP is the physician in charge of directly caring for patient and responsible for referrals to specialists.

Data Flow

Refers patient for screening—via electronic message, fax, or written prescription sheet

May be required to provide in some of the following information:

Patient name, age, SS number,

Patient diabetes information: duration of diabetes, other associated systemic/ocular conditions, duration and Rx

Other providers to whom report should be sent (diabetologist)

Preferred ophthalmologist to be contacted by screening center

Screening Site

One or more screening sites may be physically located within primary care physician's office (if sufficient numbers of patients are screened), within diabetologists' offices, within a diabetes or general medicine clinic, within a diabetes care center (where diabetics receive other “walk-in” ancillary services or testing), or elsewhere. Also, a screening site may be mobile, traveling between physicians and care center offices. Each screening site preferably includes:

retinal (fundus) camera with CCD sensors

Computer subsystem

Image quality assessment algorithms (where the central server function are performed at the screening site)

Database:

Patient name, SS number

DM data: duration of diabetes, other associated systemic/ocular conditions, current medications (will be automatically updated and evaluated by class), BP, HgAlC

Dates of screenings

Primary care physician

Other participating physicians (diabetologists, cardiologists, nephrologists)

Ophthalmologist

Data Flow

Patients are referred to center for unscheduled (preferred) or scheduled screening

Induction report is generated first as a clip-board survey filled out by patient or as the same form previously completed by PCP and transmitted to screening center (paper, fax, electronic)

Screening center may assign to a patient a default PCP, and/or a default ophthalmologist, and/or a default diabetologist

Encourage PCP and patient to choose participating ophthalmologist

Patient undergoes undilated fundus photography of each eye (preferably between two and seven) photographs of contiguous fields by operator

Screening center system provides immediate assessment of quality of photographs (illumination, contrast, focus and positioning), guiding the photographer, suggesting dilation if appropriate.

Patient may undergo additional photography with dilation if indicated by inadequate photographs without dilation.

Screening center sends report back to PCP, diabetologist when screening accomplished; or a no-show report is patient never appeared

Show/no-show report send by paper, fax, electronic message

Screening center sends all images and data to site when RGAs are processed (optionally the screening center)

Estimate approximately 10-15 Mb per patient, 2 eyes (depending upon number of fields photographed per eye

Reminds PCP and patient to have patient screened annually (or at other determined intervals)

Retinopathy Grading Algorithms

The RGAs may be executed at the screening center CPU or may be offered as an ASP service by a central server. The RGA site/central server also stores and backs up image data, patient data, report data from ophthalmologists.

Data Flow

RGA results sent to screening center, to PCP, and to identified physicians such as a diabetologist

Recommends only repeat screening (default 1 year) if grade is 21 or less

Recommends follow-up by ophthalmologist if grade 21+

RGA sends ungradable images and images requiring follow-up to designated ophthalmologist (along with induction data material) by electronic transfer

RGA Sends reminders to participating ophthalmologist to return results of evaluation of images and at intervals of scheduled visits to return management reports of evaluations and treatment of patient (see form).

Participating Ophthalmologist

A participating ophthalmologist (PO) has credentials for evaluating/treating diabetic retinopathy and agrees to review images and data submitted within agreed interval and to return evaluation sheet and/or evaluation/ management report.

Data Flow

PO receives images for grading evaluation

PO sends electronic image evaluation report back to RGA for images evaluated

PO sends electronic evaluation/management report back to RGA after patient seen.

Non-Participating Ophthalmologist

A patient or a PCP may request that reports go to an optometrist or ophthalmologist who is not participating in the OSS but cares for the patient as a specialist. For example, the patient may move to an area where the OSS is not available. It is preferable for patients and PCP to work with participating ophthalmologists.

Data Flow

The reports of screening, and if requested the images will be transferred via paper to the non-participating ophthalmologist/optometrist.

The RGA will send requests for information along with the image evaluation report form

5.3.3. Retinopathy Grading Algorithms

The Retinopathy Grading Algorithms (RGA), executed preferably within the Central Server, are one of the core elements of an OSS system. RGAs include image processing algorithms that are capable of accurately detecting and identifying in fundus images the lesions and features characteristic of various retinopathies. Based on quantitative analysis of the properties of identified lesions, an additional processing layer arrives a numerical grade level compactly characterizing the detected retinopathy. A preferable system grading scheme includes three levels used principally by the WFM to manage system processing as described above. Theses levels include: level 1, no retinopathy; level 2, retinopathy present but not currently significant; level 3 significant retinopathy currently present. More preferably, the RGAs also return a grade corresponding to clinical grading system in use for the various retinopathies, the clinical grades then being easily related to the system grades where necessary.

RGA results from complete evaluation of all fundus images are stored in the Central Database. Preferably, RGA results include evaluated image annotated or marked-up with indicia to identify, e.g., the position or the identity of detected lesions. In cases of doubt, the annotation may include indications of “definitely a lesion,” or “possibly a lesion.” Annotations can include highlighting, coloring, outlining, pointing with arrows or the equivalent, and other methods known in the art (such as text superimposed on the image). Color coding of lesion characteristics may be used to simplify interpreting the annotations. The annotated images are saved (using an appropriate naming convention) along with the original images in the CDB.

In the case of ungradable images of images having significant disease, a trained ophthalmologist is sent the images electronically for manually evaluation and grading. When a human grading report is received by the Central Server, it is automatically routed by the WFM to the patient's physicians. Upon completion of the expert grading process, all grading reports and annotated images produced by the expert are sent to the CDB for storage along with any regular grading report.

5.3.3.1. Grading Algorithm Principles

The RGAs are based on detecting and identifying “lesions” in fundus images. Therefore, each image (field of view) is evaluated to detect the number and type of lesions, and the cumulative lesion information for all acquired images is processed to arrive at a final retinopathy grade level for each eye. This processing may be by an expert system, perhaps rule-based, that simulates the considerations of an ophthalmologist when presented with similar cumulative lesion information.

Herein, and in this application generally, the term “lesion” should be carefully understood to mean identifiable visual features sought for by an ophthalmologist in order to evaluate retinal disease. For example, certain retinopathies are characterized by the presence of visually discrete features with determinable boundaries that appear more or less abruptly in time. DR is such a retinopathy which can be evaluated in terms of its associated, well-known features, including dot, blot, and striate hemorrhages, lipid exudates, nerve-fiber-layer infarcts, and so forth. Other retinopathies, however, are characterized by more diffuse visual changes. For example, age-related macular degeneration (ARMD) is characterized by diffuse alterations in retinal pigmentation—hypopigmentation or hyperpigmentation—appearing gradually with age. Thus the term “lesion” signifies visual features more general than the discrete feature often the subject of the arts of image processing.

Therefore, in order to ensure a high degree of specificity and sensitivity in detecting the wide range of features that may appear in fundus images of various retinopathies, the RGAs of this invention (90 in FIG. 3B) preferably employ iterative, top-down image processing techniques. FIG. 3C illustrates a preferable RGA implementation suitable for a wide variety of retinal conditions.

The highest level of RGA processing is illustrated in FIG. 3C at steps 100-102. Step 100 represents the determination by the image quality algorithms that a set of acquired images is suitable for RGA grading. Step 101 processes each image to detect and identify ophthalmologic lesions (as just defined above) and returns lesion-by-lesion information including lesion type, lesion size, lesion location, and so forth. Steps 103-111 further describe lesion processing. Finally, step 102 uses all the lesion information returned from step 101 to arrive at an overall retinopathy grading and evaluation. For example, this step may be implemented as an expert system that simulates the reasoning of an ophthalmologist presented with the accumulated lesion information. Thus, grading rules may be executed in view of the accumulated lesion information.

The next level of RGA processing is illustrated by steps 103-105 and their substeps. Step 103 process an image with more simple and more general image operators 106, such as local filters for smoothing or edge enhancement, thresholding to identify significant combinations of such simple features, and so forth, and returns an image marked up with the location of regions potentially having the lesions of interest 107. Step 104 then examines more complex aspects of the identified regions image. Here, it is useful to evaluate the shape and geometry of each marked-up region 108; is it compact or extended, is it located near anatomic landmarks in the retina, is it related to other marked-up regions, and so forth. Regions not meeting geometric criteria for the lesion of interest are then dropped 109 from further processing. Finally, step 105 performs the most detailed and expensive image processing but limited to determining signatures, which are lists of image parameters, attached to each of the regions of greatest interest. Signatures can include, i.e., detailed isotropic or directional texture characteristics, spectral properties such a hue and saturation, and so forth.

Finally in step 101, the signatures of the most interesting regions are examined to select, detect, and identify lesions. This selection process may, in some cases, simply rely on fixed boundaries defined in signature-parameter space. In other cases, an expert system may mimic the qualitative judgment made by a ophthalmologist reviewing the same image. In still further cases, various classification methods may be used. For example, neural networks or Bayesian classifiers may be trained on the accumulated images in the CDB to classify signatures into lesions.

It has been discovered, that centralized storage of retinal images provides an invaluable means for continuous improvement of the grading algorithms. Preferably, the means for improvement can be automated with learning methods such as, for example, genetic algorithms or neural networks. Further, this invention provides the above mechanism for improvement of algorithms through reviews, reiterative evaluation, and testing. The grading process of the present invention has been specifically automated with the objective of comparison of lesion data with as large a database as possible of prior data. An additional objective of this invention is to provide for a reduction in cost of the retinal screening.

Finally, RGAs have been discovered to be dependent on camera properties, digital image pixel density, depth and the magnification, and so forth. This dependence is preferably factored into RGA processing, for example, by inverse transforming known effects from the image.

Use of Spectral Information

Spectral information can provide important information in discriminating retinal leasions and features during the image processing phases of RGAs. The following presents the color and spectral characteristics of several types of retinal lesions. Green and yellow-green wavelengths enhance identification of the vessels and hemorrhagic lesions in the retina against background, because of peak absorption in this wavelength with a nearly maximal difference between saturated oxyhemoglobin in arteries and desaturated hemoglobin in veins. Therefore, use of these wavelengths is important in DR screening.

However, use of these wavelengths can cause difficulty in differentiating hemorrhagic lesions from hyperpigmented lesions in the retinal pigmented epithelium (e.g. laser scars or other scars), and also in differentiating lipid exudates from drusen or from nerve-fiber layer infarcts. Hemorrhagic lesions may be separated from retinal pigment epithelial lesions (hyper pigmentation) by using lesion size and texture evaluation in combination with luminosity ratios against the diffuse background luminosity within the color domains of known particular lesions. For example, hemorrhagic lesions are darkest at 535-555 nm, while retinal pigment epithelial scars are darkest at 590-620 nm.

Retinal nerve-fiber-layer striations, which are important to detect in glaucoma, are best identified at 450-495 nm which hides much the underlying, confusing vessel patterns. Nerve-fiber-layer infarcts, which are pale white to slightly cream, are difficult to differentiate from drusen and from lipid exudates, which are more cream to yellow or pale brown, but their separation may can be enhanced by utilizing color domain information in the form of luminosity contrast ratios of the suspected lesion against the background luminosity within the wavelengths that are indicative of the suspect lesion types. Lipid exudates can be best separated in the yellow-orange wavelengths that identify drusen from blue-green maximum for nerve-fiber-layer infarcts.

Precise color information for lesions of various types is best obtained from images carefully screened by retinal experts. Such screened lesions are collected in a portion of the CDB and used for training RGAs within. Further, precise lesion color and background pigmentation varies with ethnic background, being different on average in Caucasian, Hispanic or Afro-American fundi. Therefore, lesions identified by experts and stored in a training database preferably provide a variety of appearances for each lesion as observed in the fundi of different ethnic backgrounds.

5.3.3.2. RGA for Diabetic Retinopathy

As the major cause of blindness in the developed Western world, diabetic retinopathy (“DR”) may not be reversible, but the devastating and permanent effects of this disease can be prevented with early detection and treatment. An OSS system has important and demonstrated advantages in managing this ocular condition.

A preferable RGA directed to DR evaluates (note that diagnosis is not a current goals of this invention) screens diabetic eyes into three standard grades of retinopathy: no retinopathy (DR grade or OSS grade 1); micro-aneurysm alone (DR grade 21 or OSS grade 2); and micro-aneurysm with other lesions (dot and blot hemorrhages, striate hemorrhages, nerve fiber layer infarcts, or lipid exudates) (DR grade 21+ or OSS grade 3). Grade 10 (1) patients are recommended should return in, e.g., 1 year for a routine annual screening. Grade 21 (2) patients are recommended to return earlier, especially if there are other, non-ophthalmic, risk factors in their disease history (such as elevated HgbAlC). Grade 21+ (3) patients are recommended to promptly see an ophthalmologist for careful follow-up or treatment. Also for Grade 21+ patients, their retinal photographs, or any photographs that are deemed ‘ungradable’ because of poor quality, are electronically transmitted to a participating referral ophthalmologist, who reviews them and replies electronically with impressions and recommendations, including whether examination or re-screening at a shortened interval are indicated. Hence, specialists will be occupied only with those patients who require careful evaluation and treatment.

The preferably three level automatic RGA screening has been demonstrated solid clinical foundations. First, the more severe, potentially sight threatening stages of retinopathy, such as macular edema or neovascular proliferation, do not occur without the accumulation of at least some of these earlier lesions. (Klein et. al., 1997, Arch. Ophthalmol. 115:1073-1074; Klein et al., 1989, Arch. Ophthalmol. 107:1780-1785) Also, less than 20% of large population of diabetics have grade of 21+ retinopathy. (Klein et al., 1984, Arch. Ophthalmol. 102:520-526: Klein et al., 1984, Arch. Ophthalmol. 1984;102:527-532.) Thus, screening by photography and evaluation by the OSS system, on average, is estimated to remove approximately 80% of those patients who do not need more careful evaluation or treatment by a specialist.

The these RGA algorithms are directed to determining these generally discrete and well-circumscribed lesions. Further, because peripheral lesions rarely occur without central lesions, these algorithms are directed to processing images of the central retina about the optic nerve head and the fovea. Since DM is a disease that prominently affect micro-vasculature, DR algorithms preferably process green filtered (535 nm wide band pass interference filter) or yellow-green filtered, images. Because of peak absorption in this wavelength with a nearly maximal difference between saturated oxyhemoglobin in arteries and desaturated hemoglobin in veins, vessels and hemorrhagic lesions in the retina are enhanced against the background. All images have a resolution of at least 1024×1024 resolution with an 8-bit depth.

However, certain information is lost in mono-spectral processing. For example, using only green wavelengths, it has been found difficult to differentiate hemorrhagic lesions from hyperpigmented lesions (e.g. laser scars or other scars) in the retinal pigmented epithelium, or to in differentiate lipid exudates from drusen or from nerve-fiber layer infarcts.

FIG. 3D illustrates an exemplary implementation of RGA 120 for DR. Here, the images are processed in order of increasing retinopathy grade so that unnecessary processing may be avoided. First, each image is processed 121 to detect micro-aneurysms. If none are found, the image is grade 10. Next, if micro-aneurysms are present, each image is processed 122 to detect hemorrhages, such as blot hemorrhages. If no hemorrhages are find, the image is grade 20. Finally, if any further lesions are found in processing 123, the grade is promoted to 21+.

In the exemplary implementation of FIG. 3D, the conceptually distinct steps of lesion-specific processing and decision function processing illustrated in FIG. 3C are combined for processing efficiency. Therefore, in the conceptual scheme and arrangement of FIG. 3D, DR grading proceeds with lesion-specific processing which detects micro-aneurysms, hemorrhages such as blot hemorrhages, and other lesions. The decision function simply assigns grade if no lesions are found, grade 20 if only micro-aneurysms are found, grade 21 if micro-aneurysms and hemorrhages are found, and grade 21+ if micro-aneurysms, hemorrhages, and other lesions are found.

In somewhat more detail, the following lists DR lesions that are preferably detected and identified by all RGA algorithms. Sophisticated RGA algorithms for DR detect additionally the advantageous lesions.

DR Lesions and Characteristics Preferably Identified

Optic nerve head

Fovea (or approximate foveal location)

Major arteries: 1^{st}, 2^{nd}, 3^{rd} order vessels

Major veins: 1^{st}, 2^{nd}, 3^{rd} order vessels

Dot hemorrhages/micro-aneurysm—number, density, distance to the optic nerve head or to the fovea in each field

Blot hemorrhages—number, size, density, distance to optic nerve head or fovea in each field

Striate hemorrhages—number, density, distance to optic nerve head or fovea in each field

Nerve-fiber-layer infarcts—number, distance to the optic nerve head or to the fovea in each field

Lipid exudates—number, size, clustering and distance to fovea in each field

DR Lesions and Characteristics Advantageously Identified

First, size and number in each field intra-retinal micro-vascular abnormalities including:

Epi-retinal (or epi-papillary) neovascularization—size and distance to optic nerve head or fovea

Intra-retinal micro-vascular abnormalities—are small clusters (about the size of nerve-fiber-layer infarcts) of striate hemorrhagic lesions (high form factor) which lie between major retinal vessels

Epi-retinal neovascularization—cluster of small rete vessels (round configuration, caput medusa) that do not pursue the normal orientation of retinal vessels may be as small as ⅓ to ½ of optic nerve head and as large as 4-5 optic nerves

Second, diameter and tortuosity measurements for major vessel abnormalities including:

Major artery tortuosity—deviations of 1^{st}, 2^{nd}, and 3^{rd} order arteries from a straight line (point-to-point); also requires determination of whether the deviations are caused by branchings or by deviations between branchings; in other words, if a vessel branches unequally (daughter vessels are unequal in caliber), this causes a deviation of the large parent vessel into the larger of the two daughter vessels

Major vein tortuosity—deviations of 1^{st}, 2^{nd}, and 3^{rd} order veins from a straight line (point-to-point) and whether deviations are caused by branchings or by deviations in between branchings

Major artery diameter (and variation in diameter) versus distance along vessel starting at the optic nerve head—for 1^{st} and 2^{nd} order vessels; second order vessels are defined as either two daughter vessels after an equal branching (branching in which both daughter vessels are of same caliber) or the smaller caliber vessel of the daughter vessels in an unequal branching

Major vein diameter (and variation in diameter) versus distance along vessel—for 1^{st} and 2^{nd} order vessels

Next are presented certain exemplary reports such as may be exchanged and stored in a system of the present invention directed to DR. These reports are merely exemplary of the information that may be useful and are not to be taken as limiting.

Exemplary Ophthalmologist/Optometrist Report

Name of ophthalmologist submitting report

Patient information

Findings

Optic discs: estimated cup/disc ratio, abnormal cupping, abnormal atrophy, abnormal vessels:

Major arteries: normal, abnormal caliber, abnormal tortuosity

Major veins: normal, abnormal caliber, venous beading, abnormal tortuosity

Micro-vasculature: no diabetic retinopathy, dot hemorrhages /micro-aneurysms—number, lipid exudates-location, nerve-fiber layer infarcts, blot hemorrhages-number, striate hemorrhages-number, IRMA, neovascularization-location, neovascular fibrosis, vitreous hemorrhage, other pathology (branch retinal vein occlusion hemorrhages, drusen—location, macular degeneration, PED, CNVM/exudate/hemorrhage,

Recommendations:

Patient should be re-screened at interval of —3 months, 6 months, 1 year, 2 years

Patient should have photographs taken with dilation

Patient will be seen in my office for examination—as soon as possible, 2 weeks-1 month, 1-3 months, 6 months

Exemplary Referring Physician Report for Diabetic Patient

Patient identification, race

Type of diabetes (insulin requiring diabetes, most recent Hg. AlC, non-insulin requiring)

Duration of diabetes

Other Associated systemic diseases—hypertension, hyperlipidemia, nephropathy, neuropathy, anemia, other

Current medications ocular conditions (cataracts, cataract surgery, glaucoma, diabetic retinopathy, macular degeneration)

Physician information (primary care physician, ophthalmologist/optometrist, diabetologist, cardiologist, nephrologist)

Exemplary Screening Center Report for Diabetic Retinopathy:

Referring physician

Patient identification

Reported for screening on date; no show to date

Results of retinal photographic screening (photographs of adequate quality, pupils required dilation to obtain adequate photographs, photographs unsuitable for grading)

Recommendations:

Discuss with the patient the importance of undergoing screening for retinopathy even though his/her vision may be normal.

Return for routine retinopathy screening after specified interval Photographs forwarded to participating ophthalmologist for evaluation and recommendation

5.3.4. Statistical Analysis

The centralized CDB architecture of an OSS system provides a resource of unparalleled power for correlating aspect of the various retinopathies in ways that have not been possible until the advent of the present invention. By analyzing longitudinal data for individual patients, quantitative histories of particular type of lesions over time will provide a truer indication of the risk for progression than the changes in overall composite grades that have been hitherto available. These analyses may be not only at the level of the overall retinopathy but also at the level of individual types within a retinopathy.

In addition, quantitative image processing of fundus images with additional software permits evaluation of other characteristics of the fundus that cannot be evaluated by qualitative human observation. Whether or not such characteristics are now known to be markers of disease or risk, their investigation may prove beneficial in establishing more clearly risks for the development of retinopathy or its progression especially in the early stages. Such characteristic include, but are not limited to, such vascular parameters as vascular diameter and variation, tortuosity, and branching angles.

In other words the present invention is not to be understood as limited to detecting known lesions in retinal images. But as part of routine processing, an OSS can accumulate data on other retinal characteristics that can be algorithmically recognized which may yield new insights on the risk of retinopathy.

The following lists exemplary and non-limiting statistical information which may be obtained and accumulated in an OSS implementation. The following statistical parameters may be accumulated to aid in quality control and oversight of an OSS.

Exemplary Quality Control Statistics

Percent of eyes requiring dilation to obtain adequate photographs (correlation with age)

Percent of eyes with inadequate quality photographs (correlation with age)

Percent of eyes screened (with adequate photographs) with no retinopathy, minimal, significant retinopathy

Percent of patients referred who are screened within 1 month, 3 months, 6 months, 1 year of referral

Results of patients referred to ophthalmologist:

Percent of patients who are referred to participating ophthalmologists

Percent of patients who are recommended for repeat photographic screening in 3 months, 6 months, 1 year (correlation with number of lesions)

Correlation of lesion number noted by ophthalmologist with that noted by RGA

Duration between screening and eventual treatment—correlate with level of retinopathy and lesion number

Exemplary Patient Statistics

Preferably, in addition to basic patient demographic information, the following data is collected.

Age (years)

Duration of retinopathy or primary disease (years)

Duration between registration (referral) and screening (hours)

Frequency of screening

Number of images taken of each field without dilation (number)

Number of images taken of each field with dilation (number)

Pupil size without dilation (mm)

Pupil size with dilation (mm)

Presence of cataract (yes/no)

Image Quality Assessment algorithm grading of image quality

Retinopathy level by retina specialist grading (at least three system grades and “ungradable”)

Retinopathy level by RGA grading (at least three system grades and “ungradable”)

Number of lesions of each type marked by retina specialist in each field (for example, for DR dot hemorrhages, blot hemorrhages, striate hemorrhages, lipid exudates, nerve-fiber-layer infarcts)

Number of lesions of each type detected in each field by RGA

Duration between report of recommended follow-up of level 3 eyes to specialist or ophthalmologist and the completion of follow-up; frequency of follow-up evaluation

Exemplary Population Statistics

The following exemplary information may be gathered by the population of patients being managed by an OSS implementation.

Number of eyes not requiring dilation that achieved adequate image quality for each field of view

Number of eyes requiring dilation in order to achieve adequate image quality for each field of view

Number of eyes that failed Image Quality Assessment in spite of dilation

Percent of eyes screened with each of the three grades of retinopathy

Comparison of population to prior reports of similar retinopathy

Percent of images failing Image Quality Assessment—correlation with age, pupil size, presence of cataract

Is there a high number of dilations per site tied to an individual operator

Percent of eyes with more advanced lesions noted in each field (1, 2, 3, 4 or 5) without equivalent lesions noted in other fields

Percent of patients who complied with recommendation for screening and follow-up screening; time interval between receipt of recommendation/referral by patient and actual follow-up screening

Sensitivity & specificity of the grading algorithm as compared with the gold standard of grading performed by the retinal specialist—for each eye (variance with age, pupil size, necessity for dilation, presence of cataract)

Yet to be identified additional correlation possibilities

5.3.4.1. Lesion Tracking

The systems and methods of the present invention importantly provide, for the first time to the inventor's knowledge, the ability to tack retinopathy quantitatively and lesion-by-lesion. Hitherto, human retinal evaluations have necessarily been quantitative; all retinal details have been condensed into a verbal summary, or even into a single numerical grade.

In contrast, the present invention makes available quantitative information all detected and identified lesions or for parameters of retinal vasculature Screening repeated over time then provides the time evolution of this quantitative retinal information. Comparison algorithms can automatically follow changes in the lesion characteristics, their number, or position, can by follow vascular parameters such as tortuosity, size, and beading, and thereby determine progression (or regression) of the retinopathy lesion by lesion. Population studies of progression or regression can considerably refine risk prediction for retinopathy patients. Risk prediction factors that can be quantitatively assessed for the first time include accumulation of increasing numbers of lesions, grouping of lesions, positional progression towards key structures such as the fovea or the optic nerve head.

Further, in certain retinopathies, lesion detection itself may require tracking retinal image characteristics. For example, the pigmentation changes in age-related macular degeneration can only be certainly identified if they are observed to expand or change with respect to prior or baseline images. Thus, the present invention provides for the first time for certain quantitative assessment of such retinopathies.

In a preferred (but exemplary embodiment), lesions, and image features generally, may be tracked over time by comparing their positions relative to retinal landmarks known to be relatively fixed in position. Since the retina is not necessarily fixed over time, care is required in choosing such fixed landmarks. Fixed landmarks include of course the optic nerve head, the fovea, which however are rather large. Further fixed (or “invariant”) features are crossing and branching points of retinal vessels, especially major vessels. However, points on vessel in between crossings and branchings may not be fixed because vessels may increase in tortuosity over time. Other fixed points that may occur in certain cases may be used in the lesion-tracking methods described.

FIG. 3E illustrates a preferred method for tracking and correlating lesions and features over time. First, images of corresponding fields 131 in a patient are selected from screenings performed at two different times 130a and 130b. Then the “invariant” features to be used for image matching are selected and recognized in both images, for example, major retinal vessels. Several corresponding points are chosen on these features in both images to define a spatial and positional image matching 133 between the two images. If the defined matching is within tolerance, for example, by mapping remaining feature points with precision and not unreasonably mapping any part of the retina, and if all the image can be so matched, then the transformed images are compared 135. This comparison can then quantitatively evaluate intrinsic lesion and feature changes independently of global changes in the retina. Finally, a report is generated 136, stored in the CDB, and transmitted to relevant physicians. Further, progression and regression information can be incorporated into an expanded grading system that looks beyond only the current appearance of the retina.

Lesion tracking may also be an essential part of quality control and RGA development. For example, lesions are identified by an expert on digital images and the centroid (or other position indicator) is painted by hand, optionally in a color coded manner. A lesion that is unequivocally identified is painted with one color while those lesions that are equivocal are identified by another color. The expert-identified lesions can then be compared on an individual basis, lesion by lesion (identified by position in the retinal photograph), with the lesions identified by RGA processing. Discrepancies may be then used to improve the grading algorithms.

5.3.5. Data Retention

An OSS CDB is most preferably configured with storage and backup processes so that all data necessary data for legal, regulatory, and commercial requirements are saved for at least the time periods required. Further, backup processes are advantageously structured to be flexible to alter their policies to track changed requirements.

5.4. Physician Access

As illustrated in FIG. 1A, physician access is of two general types; one is office access by primary care physicians (“PCP”), specialists (such as diabetologists), and ophthalmologists that do not participate in the OSS, and the other is office access by participating ophthalmologists (“PO”). Physician access of the first type has relatively modest requirements. In a preferred embodiment, any system with a web browser and e-mail capabilities is sufficient, such as a PC-type or Macintosh-type personal computer with Netscape Communicator or Microsoft Internet Explorer.

The primary care physicians and specialists who treat patients screened by an OSS system need to access screening results and reports As well as downloading the reports and screening results, they receive message communicating the tracking of the patient through the system. For example, if a patient was recommended for screening, the referring physician would be notified if the patient had not been screened within the OSS network after a specified period of time had elapsed. The physician is also provided a mechanism via a template/form to make a system referral or to add notes to the patient's ‘folder’ that is stored with the reports, screening results, etc in the OSS system. These function are preferably provided by interaction with OSS application according to an ASP model. In one embodiment, some or all of the information generated by a PO can be exchanged in a structure format, for example, using XML forms.

5.4.1. Ophthalmologist Access

Access for participating ophthalmologists requires additional features beyond those for specialists or PCPs. Because a PO exchanges image data with the central server—both images sent automatically for evaluation, images requested as needed, and evaluated and annotated images returned to the server—an adequate system must provide adequate communication bandwidth to the central server along with display, storage, and processing capabilities for evaluation and annotation of high resolution messages. A PO system also provides facilities for message exchange and for report generation and exchange.

In more detail, FIG. 4 illustrates the OSS methods transpiring in a PO office. Image are received from the central server at least because they are in inadequate quality to be automatically graded (as indicated by off-page connector 140a) or because the have been automatically graded and found to have significant retinopathy (as indicated by off-page connector 140b) (for example of system grade level 3) requiring specialist review. These images are then reviewed 141, and annotated images are optionally returned for storage in the CDB (as indicated by off-page connector 147a). The PO next determines whether the patient should either be examined 142 or re-screened 143 at a shorter, and a final report is returned to the centra server (as indicated by off-page connector 147b).

If the PO determined that examination is recommended, the WFM looks for a series of events to determine if the patient appeared for examination 144 (at the PO or at another ophthalmologist), and if the examination report has been generated 145 and transmitted to the central server. The WFM also checks the examining ophthalmologist recommendations concerning further examination, screening, or treatment, and schedules the necessary events. The WFM also routinely (preferably electronically) informs the patient's direct care physicians of the results of these examinations.

5.5. Security, Privacy, and Integrity

Data integrity and the secure access of patient data are of the utmost importance. In the United States the HIPAA (Health Insurance Portability and Accountability Act) and associated regulations govern medical data; corresponding laws and regulations exist in Europe and in other jurisdictions. Systems of the present invention are designed to provide the system wide architecture needed to address all such security issues in order to verify data integrity, protect the data from unauthorized usage and to ensure patient confidence and confidentiality. Through the system components chosen that support security as well as the proper design, implementation, and operation of the system infrastructure, the OSS architecture ensures compliance with laws and regulations. In preferred embodiments, the following facilities are provided.

1. Authentication—provide assurance that each user or system entity is who he/she/it claims to be; authentication must be performed at multiple levels in order to ensure the security of enterprise architecture:

User authentication—unique user identification via login/password; i.e., confirm user is a member of the OSS PROJECT or approved guest

Entity authentication—Each computer in the OSS network has a static, known/trusted IP address. As all communication between the systems travels over a network, all data shall be encrypted in transit.

Message authentication—Each transaction shall incorporate the use of digital certificates as verification of origin of the message.

Automatic logoff—If the user ‘walks away’ from the remote workstation the connection to the Central Server is severed after a designated period of time in order to prevent unauthorized access to the system.

2. Access control/authorization—the mechanism used to grant or deny access to and disclosure of medical information belonging to a specific patient; i.e., does this user have permission to access this patient's data; thus the privacy and confidentiality of the patient medical record is ensured; one or more of the following methods is used in different embodiments:

Identity-based—access based on user name/password login

Role-based—access based on the role of the user within an organization

Context-based—access based on the contextual relationship between user/health care provider and the patient

Physical safeguards—The Central Server is placed in a locked room with restricted access allowed only to authorized personnel. Each of the remote sites (screener subsystem and physician offices) must have policies and procedures for limiting physical access to an entity while ensuring that properly authorized access is allowed.

3. Integrity Protection—protection of external communications and remote access points as well as the transfer and storage of data is handled by multiple levels of security:

Firewalls—As OSS is a geographically distributed network, each site has it's own firewall in order to restrict access to the medical data contained on the workstation. The Central Server firewall limits system connections to only members of the OSS network based on static, known/trusted IP addresses.

All firewall software is industry standard COTS (Commercial Off The Shelf) products, and shall not hinder physician's workstations in accessing the web or email communications.

Transit Encryption—All transactions shall be implemented using a combination of secure technologies to ensure transit security Secure Socket Layer ‘SSL’ protocol is used to create a secure connection between a client and server in combination with Secure HTTP (S-HTTP) protocol that is designed to transmit individual messages securely. Digital certificates issued by a ‘Certificate Authority’ shall be used as the key in the PKI (Public Key Infrastructure) to authenticate the validity of each party in the Internet transaction. Data is encrypted using a ‘COTS’ encryption package in accordance with HIPAA standards (128 bit encryption is applied, at a minimum)

Database Encryption—The database management system has ‘Triple DES Encryption’ capability to ensure that even one who has super-user/database administrative privileges cannot access the parts of the database containing confidential medical data. This functionality is implemented if so required by HIPAA regulations.

Virus Detection—Use of commercial virus detection software as well as cryptographic seals such as checksums, and hash functions is employed.

Disaster recovery—A comprehensive plan is in place to ensure the ability of the OSS System to be rebuilt in the case of a system crash. Both RAID and tape backup is used in the disaster recovery plan of the Central Server. Each screening site archives the patient's images and associated data on CD's for storage within the institution.

4. Attribution (Non-repudiation)—assures that information that is said to be from a specific user or system are as claimed, i.e., provides proof that the sender sent and the receiver received.; mechanisms providing attribution shall be implemented via commercially available products:

Digital certificates

Intrusion detection software

Audit trails—provides attributable record of system events that have transpired . . . knowledge of who has accessed which patient files

The invention described and claimed herein is not to be limited in scope by the preferred embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

A number of references are cited herein, the entire disclosures of which are incorporated herein, in their entirety, by reference for all purposes. Further, none of these references, regardless of how characterized above, is admitted as prior to the invention of the subject matter claimed herein.

What is claimed is:

1. A method for acquiring one or more digital retinal images of adequate objective quality from a patient during a single image acquisition session, the method comprising:
acquiring a digitally-encoded photographic image of a retinal field in an eye of the patient with a retinal camera,
determining one or more objective quality measures for the acquired digitally-encoded image by processing the image with one or more image quality assessment algorithms, wherein the image is determined to be of adequate quality if all the objective quality measures are determined to be adequate,
repeating the steps of obtaining and determining only if one or more of the determined quality measures are determined to be inadequate,
wherein, prior to repeating the step of obtaining, instructions are provided to adjust the retinal camera in a fashion to correct inadequate quality measures, and
wherein the repetitions, if any, of the steps of obtaining and determining are limited by the duration of the image acquisition session.

2. The method of claim 1 wherein the step of repeating is limited to at most three repetitions of the steps of obtaining and determining.

3. The method of claim 1 wherein the one or more objective quality measures determined by the image quality assessment algorithms are correct image orientation, or correct level of image contrast, or correct image focus, or absence of image edge flare

4. The method of claim 3 wherein (i) if image orientation is inadequate, then the provided instructions comprise visual mis-alignment examples and corrective actions relating to the relative rotation of the camera and the eye,
(ii) if image contrast is inadequate, then the provided instructions comprise corrective actions relating to the relative anterior-posterior position of the camera and the eye,
(iii) if image focus is inadequate, then the provided instructions comprise corrective re-focusing actions, and
(iv) if absence of image edge flare is inadequate, then the provided instructions comprise corrective actions relating to the relative X-Y position of the camera and the eye.

5. A system for acquiring one or more digital retinal images of adequate objective quality from a patient during a single image acquisition session, the system comprising:
a retinal camera,
a computer including a processor and memory which is coupled to the camera for image transfer to the memory, and wherein the memory is provided with instructions encoding the steps of
receiving into the memory from the camera a digitally-encoded photographic image of a retinal field in an eye of the patient,
processing the image with one or more image quality assessment algorithms which determine one or more objective quality measures for the image, wherein the image is determined to be of adequate quality if all the objective quality measures are determined to be adequate, and
repeating the steps of obtaining and determining only if one or more of the determined quality measures are determined to be inadequate, such that (i) wherein, prior to repeating the step of obtaining, instructions are provided to adjust the retinal camera in a fashion to correct inadequate quality measures, and (ii) wherein the repetitions, if any, of the steps of obtaining and determining are limited by the duration of the image acquisition session.

6. The system of claim 5 wherein the one or more objective quality measures determined by processing the image with quality assessment algorithms are correct image orientation, or correct level of image contrast, or correct image focus, or absence of image edge flare

7. A computer program product for acquiring one or more digital retinal images of adequate objective quality from a patient during a single image acquisition session, the product comprising at least one computer-readable memory with encoded instructions for
receiving into a memory of a computer from a camera a digitally-encoded photographic image of a retinal field in an eye of the patient,
processing the image with one or more image quality assessment algorithms which determine one or more objective quality measures for the image, wherein the image is determined to be of adequate quality if all the objective quality measures are determined to be adequate, and
repeating the steps of obtaining and determining only if one or more of the determined quality measures are determined to be inadequate, such that (i) wherein, prior to repeating the step of obtaining, instructions are provided to adjust the retinal camera in a fashion to correct inadequate quality measures, and (ii) wherein the repetitions, if any, of the steps of obtaining and determining are limited by the duration of the image acquisition session.

8. An automatic method for grading one or more digitally-encoded images of a retinal field of an eye of a patient with respect to a selected retinopathy, the method comprising:
processing the digitally-encoded retinal image to detect, identify, and characterize in the retinal image lesions from a pre-determined set lesion types, wherein the pre-determined set of lesion types describe visual features characteristically found in retinas with the selected retinopathy,
performing a decision process that assigns a grade to the retinal image in dependence of on properties of the detected lesions.

9. The method of claim 8 wherein the retinopathy is diabetic retinopathy, wherein the pre-determined lesion types include micro-aneurysms, or dot hemorrhages, or blot hemorrhages, or striate hemorrhages, or nerve fiber layer infarcts, or lipid exudates, or neovascularization, or intra-retinal micro-vascular abnormalities (IRMA), or venous beading.

10. The method of claim 9 wherein the decision process assigns (i) a first grade if no lesions are detected, (ii) a second grade if only one or more micro-aneurysms are detected, (iii) a third grade if one or more micro-aneurysms and one or more of dot hemorrhages or of blot hemorrhages or of striate hemorrhages are detected, and (iv) a fourth grade if one or more micro-aneurysms and one or more of dot hemorrhages or of blot hemorrhages or of striate hemorrhages and one or more of nerve fiber layer infarcts or of lipid exudates or of cotton wool spots or of neovascularization.

11. The method of claim 8 wherein the step of processing further comprises:
detecting potential lesions as identified image features not discriminated as normal retinal features,
detecting probable lesions as detected potential lesions with geometric configurations and pixel variability thresholds fitting a type of pre-determined lesion,
detecting lesions by a decision process based on image features, geometric configurations, pixel variability thresholds, and signature features of the detected probable lesions, wherein the signature features include texture parameters and spectral characteristics.

12. The method of claim 8 wherein, for the step of performing, the properties of the detected lesions comprise their identities, their numbers, their sizes, and their retinal positions.

13. The method of claim 12 wherein the retinal positions comprise positions with respect to the optic nerve head and the fovea.

14. The method of claim 8 wherein the steps of processing and performing include one or more decision processes, and wherein the method further comprises a step of training the decision processes including:
assigning grades to the plurality retinal images from patients having the selected retinopathy by performing a manual grading method,
assigning grades to a plurality retinal images from patients having the selected retinopathy by performing the automatic method of claim 7, and
adjusting the decision processes so that the grades assigned by the automatic method are of adequate accuracy in comparison to the grades assigned by the manual method.

15. A system for grading one or more digitally-encoded images of a retinal field of an eye of a patient with respect to a selected retinopathy, the system comprising:
a computer including a processor and memory wherein the memory is provided with a digitally-encoded retinal image, and wherein the memory is further provided with instructions encoding the steps of
detecting, identifying, and characterizing lesions in the digitally-encoded retinal image from a pre-determined set of lesion types, wherein the pre-determined set of lesion types describe visual features characteristically found in retinas with the selected retinopathy, and
executing a decision process that assigns a grade to the retinal image in dependence of on properties of the detected lesions.

16. The system of claim 15 wherein the instructions encoding the steps of detecting, identifying, and characterizing further encode the steps of
detecting potential lesions as identified image features not discriminated as normal retinal features,
detecting probable lesions as detected potential lesions with geometric configurations and pixel variability thresholds fitting a type of a pre-determined lesion,
detecting lesions by a decision process based on image features, geometric configurations, pixel variability thresholds, and signature features of the detected probable lesions, wherein the signature features include texture parameters and spectral characteristics.

17. A computer program product for grading one or more digitally-encoded images of a retinal field of an eye of a patient with respect to a selected retinopathy, the product comprising at least one computer-readable memory with encoded instructions for
detecting, identifying, and characterizing lesions in a digitally-encoded retinal image from a pre-determined set of lesion types, wherein the pre-determined set of lesion types describe visual features characteristically found in retinas with the selected retinopathy, and
executing a decision process that assigns a grade to the retinal image in dependence of on properties of the detected lesions.

18. A method for grading one or more digitally-encoded images of a retinal field of an eye of a patient taken at a selected time with respect to a selected retinopathy, the method comprising:
processing the digitally-encoded retinal image taken at the selected time to detect, identify, and characterize in the retinal image lesions from a pre-determined set lesions type, wherein the pre-determined set of lesion types describe visual features characteristically found in retinas with the selected retinopathy,
processing at least one digitally-encoded retinal image of the patient taken at least one time prior to the selected time to detect, identify, and characterize in the prior retinal images lesions from the pre-determined set lesions type,
comparing the lesions detected in the image taken at the selected time with the lesions detected in the prior image to detect changes in the lesions, and
performing a decision process that assigns a grade to the retinal image taken at the selected time in dependence on the identities and characteristics of the lesions detected in that image, and in dependence on the changes in the lesions detected in the comparing step.

19. A system for grading one or more digitally-encoded images of a retinal field of an eye of a patient taken at a selected time with respect to a selected retinopathy, the system comprising:
a database including at least one digitally-encoded retinal image of the patient taken at at least one time prior to the selected time,
a computer including a processor and memory which is coupled to the database and wherein the memory is provided with a digitally-encoded retinal image, and wherein the memory is further provided with instructions encoding the steps of
detecting, identifying, and characterizing lesions in the digitally-encoded retinal image taken at the selected time from a pre-determined set of lesion types, wherein the pre-determined set of lesion types describe visual features characteristically found in retinas with the selected retinopathy,
retrieving into memory the digitally-encoded retinal image of the patient taken at the prior time,
detecting, identifying, and characterizing lesions in the retrieved retinal image taken at the prior time from the pre-determined set lesions type,
comparing the lesions detected in the image taken at the selected time with the lesions detected in a prior image to detect changes in the lesions, and
performing a decision process that assigns a grade to the retinal image taken at the selected time in dependence on the identities and characteristics of the lesions detected in that image, and in dependence on the changes in the lesions detected in the comparing step.

20. An automatic method for annotating one or more digitally-encoded images of a retinal field of an eye of a patient with respect to a selected retinopathy, the method comprising:
processing a digitally-encoded retinal image to detect, identify, and characterize in the retinal image lesions from a pre-determined set lesion types, wherein the pre-determined set of lesion types describe visual features characteristically found in retinas with the selected retinopathy,
annotating the retinal image with indicia indicating at least the positions of the detected lesions.

21. The method of claim 20 wherein the annotation further indicates characteristics of the detected lesions.

22. The method of claim 20 further comprising:
retrieving the retinal image to be processed from a database of retinal images prior to the step of processing, and
storing the annotated retinal image in the database subsequent to the step of annotation.

23. The method of claim 22 further comprising prior to the step of retrieving:
receiving the retinal image to be processed from a source of retinal images, and
storing the retinal image to be processed in the database.

24. A computer database comprising one or more computer readable media with a database constructed according to the method of claim 23.

25. A method for managing the retinal screening of a patient likely to have a retinopathy comprising:
receiving at least one digitally-encoded retinal image taken from the patient,
receiving a grade for the retinal image from automatic retinal grading methods scheduled to evaluate the received retinal image,
performing a decision process according to which
if the grade indicates the presence of significant retinopathy, then receiving a further grade for the retinal image from manual grading methods scheduled to evaluate of the retinal image, or
if the grade indicates the presence of retinopathy but not significant retinopathy, then scheduling to receive at least one retinal image taken from the patient after a selected first interval, or
if the grade indicates the presence of retinopathy but not significant retinopathy, then scheduling to receive at least one retinal image taken from the patient after a selected second interval.

26. The method of claim 25 wherein the step of receiving further comprises
acquiring the retinal image from a retinal camera, and
evaluating by image quality assessment algorithms whether the image's quality is adequate for the automatic retinal grading methods.

28. The method of claim 27 wherein, if the received image is indicated to have an inadequate quality for the automatic retinal grading methods, then further performing a step of receiving a grade for the retinal image from manual grading methods scheduled to evaluate of the retinal image.

29. The method of claim 26 wherein the first interval is selected in dependence on the severity of the retinopathy indicated by the grade, and wherein the second interval is selected to be longer than the first interval.

28. The method of claim 25 further comprising transmitting a reminder message if a grade has not been received from scheduled manual grading methods with a selected time period.

29. The method of claim 25 further comprising
receiving a referral message from a health care professional requesting screening for the patient,
scheduling receipt of a retinal image taken from the patient, and
transmitting a reminder message if an image has not been received with a selected time period.

30. A system for managing the retinal screening of a patient likely to have a retinopathy comprising:
a database,
a computer including a processor and a memory which is coupled to the database and enabled to receive digitally-encoded retinal images, wherein the memory is further provided with instructions encoding the steps of
(i) receiving into the memory at least one digitally-encoded retinal image taken from the patient,
(ii) scheduling automatic retinal grading methods scheduled to evaluate the received retinal image, the automatic retinal grading methods returning a grade for the retinal image,
(iii) performing a decision process according to which
if the grade indicates the presence of significant retinopathy, then receiving a further grade for the retinal image from manual grading methods scheduled to evaluate of the retinal image, or
if the grade indicates the presence of retinopathy but not significant retinopathy, then scheduling receipt at least one retinal image taken from the patient after a selected first interval, or
if the grade indicates the presence of retinopathy but not significant retinopathy, then scheduling receipt at least one retinal image taken from the patient after a selected second interval, and
(iv) storing in the database the received retinal image, information returned from the automatic retinal grading methods, and information generated by the performed decision process.

31. The system of claim 30 wherein the received retinal image is taken at a selected time, wherein the database stores at least one digitally-encoded retinal image of the patient taken at at least one time prior to the selected time, and wherein the instructions encoding the automatic retinal grading methods encode the steps of
detecting, identifying, and characterizing lesions in the digitally-encoded retinal image taken at the selected time from a pre-determined set of lesion types, wherein the pre-determined set of lesion types describe visual features characteristically found in retinas with the selected retinopathy,
retrieving into memory the digitally-encoded retinal image of the patient taken at the prior time,
detecting, identifying, and characterizing lesions in the retrieved retinal image taken at the prior time from the pre-determined set lesions type,
comparing the lesions detected in the image taken at the selected time with the lesions detected in the prior image to detect changes in the lesions, and
performing a decision process that assigns a grade to the retinal image taken at the selected time in dependence on the identities and characteristics of the lesions detected in that image, and in dependence on the changes in the lesions detected in the comparing step.

32. The system of claim 30 further comprising one or more systems according to claim 5, wherein the system according to claim 5 are enabled to transmit the retinal images to the computer

33. The system of claim 30 further comprising one or more access means for health care professionals, wherein the access means provide for receipt of reports and for transmission of requests concerning the patient by health care professionals.

34. The method of claim 8 wherein the retinal image includes information at two or more wavelengths, and wherein the step of processing detects, identifies, and characterizes lesions in the retinal image with wavelength-dependent properties in dependence on the wavelength information.

35. The method of claim 1 wherein the retinal camera is a non-mydriatic retinal camera.

36. The system of claim 5 wherein the retinal camera is a non-mydriatic retinal camera.

37. The method of claim 26 wherein the retinal camera is a non-mydriatic retinal camera.
